(19) United States
(12) Patent Application Publication
(10) Pub. No.: US 2017/0080255 A1
(43) Pub. Date: Mar. 23, 2017

(54) THIN AND WEARABLE ULTRASOUND PHASED ARRAY DEVICES

(71) Applicant: CEREVAST MEDICAL INC., Redmond, WA (US)

(72) Inventors: Wing Law, Redmond, WA (US); Tomokazu Sato, Redmond, WA (US); William J. Tyler, Redmond, WA (US)

(21) Appl. No.: 15/126,272

(22) PCT Filed: Mar. 16, 2015
(86) PCT No.: PCT/US15/20839

§ 371 Date: Sep. 14, 2016

Related U.S. Application Data
(60) Provisional application No. 61/953,746, filed on Mar. 15, 2014.

(51) Int. Cl. A61N 7/00; H01L 41/09; B06B 1/06

(52) U.S. Cl. CPC A61N 7/00; B06B 1/0622; H01L 41/09; A61N 2007/0026; A61N 2007/0095

(57) ABSTRACT

Ultrasound phased array apparatuses (including systems and devices) and methods for making and using them. These apparatuses may be thin, and lightweight, so that they may be worn on a subject's head or other body region in acoustic communication so as to deliver ultrasound for stimulation of tissue, and particularly for neurostimulation. The ultrasound phased array apparatuses may be used as wearable and lightweight neurostimulation apparatuses.

### CROSS REFERENCE TO RELATED APPLICATIONS

This patent application claims priority to U.S. provisional patent application No. 61/953,746, filed on Mar. 15, 2014 (titled “RADIAL MODE ULTRASOUND ARRAY APPARATUSES AND METHODS FOR USING THEM”), the entirety of which is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

Methods and apparatuses for the delivery of ultrasound. In particular, described herein are wearable (thin and lightweight) ultrasound delivery apparatuses and methods of using them, as well as methods and apparatuses for manufacturing such devices. The wearable apparatuses described herein may be ultrasound phased array apparatuses which operate in a predominantly radial mode.

### BACKGROUND

Ultrasound systems are used in medical applications, cleaning systems, corrosion testing and inspection, power generation, petrochemical applications, pipeline construction and maintenance, aerospace applications, and a variety of other applications and fields. Devices and systems that deliver ultrasound waves in various modes have been miniaturized to the scale of hand-held sized platforms. Compared to conventional cart-like systems, hand-held and wearable ultrasound systems and devices could increase the applicability and use of ultrasound in a wide range of fields given their relatively low cost and portability.

Despite the research to date on ultrasound arrays, existing systems and methods are lacking in at least some cases with regard to miniaturization, weight, energy efficiency, and/or wearability. Further, existing ultrasound arrays are generally cumbersome, expensive, and difficult to manufacture. Cabling and connectors for ultrasound arrays can be particularly challenging technically and expensive to manufacture. Smaller, portable, and simpler systems would be advantageous for biological, industrial, and medical applications of ultrasonic technology.

For example, traditional ultrasound arrays are too large and heavy for wearable and/or portable phased arrays. The ultrasound elements in traditional ultrasound arrays alone have a thickness of about 3 mm to 5 mm and weigh approximately 30 grams to 40 grams with a total diameter of about 1 inch to 2 inches. The weight will scale with the square of the diameter, and thus will increase rapidly for larger numerical apertures suitable for focusing in biological tissue. Also, due to the nature of the construction process, these elements must remain together, preventing the simple construction of a sparser array with gaps covering a large surface area. Sparse arrays have a number of advantages including reducing cost and complexity of electronic control circuitry, lowering thermal safety considerations, reduced weight, or a flatter form factor due to electronics and cabling fitting in between elements as opposed to being attached on top, all while maintaining a larger aperture. In addition, for traditional ultrasound arrays the thickness is by necessity increased by lenses, packaging, and the cabling to power supplies and waveform control hardware since they must be attached on top of the array, as opposed to in between array elements. The final ultrasound transducer array probe may weigh well over half of a pound, with high-tension cabling, extending to power and focusing electronics, exerting additional forces on the probe itself.

Traditional ultrasound arrays are typically manufactured by potting a large piezoelectric composite plate into a backing material or a matching layer, then dicing the ceramic plate into smaller elements. Once the elements are defined, connections are made to the elements manually or semi-automatically. Matching layers, lens, backing, front plates, electromagnetic interference (EMI) shielding, electrical connections, moisture barrier, and other mechanical structures are bonded consecutively onto the array to make the finished product. Due to the mechanical impact of the dicing step, the yield of traditional arrays is difficult to control and predict. Even a small number of mechanically damaged elements can ruin the entire array. Due to the poor yield, traditional arrays are very expensive. A one-dimensional array costs upwards of several thousand dollars while a two-dimensional array is far more expensive.

Further, traditional arrays require several days to manufacture because of the many steps to attach, glue, epoxy, bind, and encapsulate the various components by hand to the array. Special fixtures and jigs are required for each of the steps for every model of an array. Additionally, a new array design may require months to plan and execute because of the many custom fixtures required. The new glue and chemicals required for a new array often stipulates elaborate qualification steps to verify that the new glue and chemicals will be amenable to high volume production.

Further, certain applications of ultrasound phased arrays, for example biological applications, require a large aperture for the ultrasound array. The size, weight, and number of elements of an array are proportional to the aperture of the array if using traditional construction methods. Thus, expanding the number of array elements typically requires that the array size and aperture be increased as well. However, increasing the size of the array to accommodate more array elements is incompatible with miniaturizing and increasing the efficiency of ultrasound phased arrays.

One biological application of ultrasound arrays is transcranial neuromodulation with low frequency, low power ultrasound. Ultrasound can be focused to a fairly small region, advantageously inducing neuromodulation in one or more spatially restricted neural target regions. It would be helpful and useful to provide a relatively low-power ultrasound apparatus for neuromodulation applications in therapeutic and consumer applications, including in particular, wearable devices. Transcranial ultrasound neuromodulation is an advantageous form of brain stimulation due to its non-invasiveness, safety, focusing characteristics, and the capacity to vary transcranial ultrasound neuromodulation waveform protocols for specificity of neuromodulation. Implanted ultrasound arrays for neuromodulation of neural tissue in the brain, spinal cord, or peripheral nervous system may also be advantageous, without requiring systems capable of passing energy efficiently through the skull or compensating for aberrations caused by the skull.

Thus, there is a need for lightweight, thin, and cost-effective ultrasound phased array apparatuses, and methods of operating and manufacturing them. Described herein are methods and apparatuses that may address the problems and needs discussed above.

### SUMMARY OF THE DISCLOSURE

Described herein are thin and lightweight, wearable ultrasound applicators including arrays of transducer elements that may be configured for phased operation.

For example, described herein are ultrasound phased array applicator devices, the device comprising: an array of ultrasound transducer elements wherein each transducer element has a width and a thickness, and the width is greater than twice the thickness, so that it operates more in a radial mode than in a thickness mode; and control circuitry configured to operate the array of ultrasound transducer elements as a phased array allowing beamforming of emitted ultrasound signals.

An ultrasound phased array applicator device may be configured to operate between 100 kHz and 1 MHz, and may include: an array of ultrasound transducer elements wherein each ultrasound transducer element has a width of between about 0.5 mm and 2 mm and a thickness, and wherein the width is greater than twice the thickness so that it operates more in a radial mode than in a thickness mode; control circuitry configured to operate the array of ultrasound transducer elements as a phased array allowing beamforming of emitted ultrasound signals; and a printed circuit board (PCB) substrate configured as a matching layer, onto which a front side of each ultrasound transducer element of the array of ultrasound transducer elements is mounted for transmission of the emitted ultrasound signals through the PCB substrate, further wherein the array of ultrasound transducers are air backed on a back side of each ultrasound transducer element.

Another example of an ultrasound phased array applicator device configured to operate between 100 kHz and 1 MHz includes: an array of ultrasound transducer elements wherein each ultrasound transducer element has a width of between about 0.5 mm and 2 mm and a thickness, and wherein the width is greater than twice the thickness, further wherein the spacing between the ultrasound transducer elements of the array of ultrasound elements is between about 0.01 mm and 2 mm; control circuitry configured to operate the array of ultrasound transducer elements as a phased array allowing beamforming of emitted ultrasound signals; and a printed circuit board (PCB) substrate configured as a matching layer, onto which a front side of each ultrasound transducer element of the array of ultrasound transducer elements is mounted for transmission of the emitted ultrasound signals through the PCB substrate, further wherein the array of ultrasound transducers are air backed on a back side of each ultrasound transducer element; wherein the control circuitry is on the PCB substrate.

In any of these variations, the control circuitry may be configured to operate the array of ultrasound transducer elements between 100 kHz and 1 MHz. For example, the control circuitry may be configured to operate the array of ultrasound transducer elements between 100 kHz and 0.8 MHz.

The ultrasound transducer elements may have a width of between about 0.02 and 5 mm (e.g., between about 0.3 mm, 0.4 mm, 0.5 mm, etc. and 5 mm, 4 mm, 3 mm, 2 mm, 1 mm, etc.). The width may be between about 2 times and 10 times the thickness for some or all of the transducer elements. The width may be approximately half the wavelength of an ultrasound signal emitted by the device. The thickness may be 25% or less of the wavelength of an ultrasound signal emitted by the device. The spacing between the ultrasound transducer elements of the array of ultrasound elements may be between about 0.2 mm and 2 mm.

In general, any of the circuitry elements for the applicators, including the control circuitry, may be printed onto a printed circuit board (PCB) substrate configured as a matching layer or as part of a matching layer (e.g., including additional matching material, to provide a predetermined thickness, e.g., ¼ wavelength), onto which a front side of each ultrasound transducer element of the array of ultrasound transducer elements is mounted for transmission of the emitted ultrasound signals through the PCB substrate. For example, the control circuitry is printed onto the printed circuit board (PCB) substrate configured as the matching layer. The PCB may be a flexible PCB.

The array of ultrasound transducers may generally be air backed on a back side of each ultrasound transducer element.

Any of these apparatuses may be configured as stimulation devices, including neurostimulation devices. For example, a wearable phased array ultrasound neurostimulator apparatus may include: an array of ultrasound transducer elements wherein each transducer element has a width and a thickness, and the width is greater than twice the thickness so that it operates more in a radial mode than in a thickness mode; control circuitry configured to operate the array of ultrasound transducer elements between 100 kHz and 1 MHz as a phased array allowing beamforming of emitted ultrasound signals; and an outer surface of the apparatus configured to adhesively attach to a subject's skin.

A wearable phased array ultrasound neurostimulator apparatus may include: an array of ultrasound transducer elements wherein each ultrasound transducer element has a width of between about 0.5 mm and 2 mm and a thickness, and wherein the width is greater than twice the thickness, further wherein the spacing between the ultrasound transducer elements of the array of ultrasound elements is between about 0.2 mm and 2 mm; control circuitry configured to operate the array of ultrasound transducer elements between 100 kHz and 1 MHz as a phased array allowing beamforming of emitted ultrasound signals; and a printed circuit board (PCB) substrate configured as a matching layer, onto which a front side of each ultrasound transducer element of the array of ultrasound transducer elements is mounted for transmission of the emitted ultrasound signals through the PCB substrate, further wherein the array of ultrasound transducers are air backed on a back side of each ultrasound transducer element; wherein the control circuitry is on the PCB substrate.

In some examples, a wearable phased array ultrasound apparatus includes: an array of ultrasound transducer elements wherein each transducer element has a width and a thickness, and the width is greater than twice the thickness so that it operates more in a radial mode than in a thickness mode; control circuitry configured to operate the array of ultrasound transducer elements between 100 kHz and 1 MHz as a phased array allowing beamforming of emitted ultrasound signals; and an outer surface of the apparatus configured to acoustically couple to a subject's skin.

A wearable phased array ultrasound apparatus may include: an array of ultrasound transducer elements wherein each ultrasound transducer element has a width of between about 0.5 mm and 2 mm and a thickness, and wherein the width is greater than twice the thickness, further wherein the spacing between the ultrasound transducer elements of the array of ultrasound elements is between about 0.01 mm and 2 mm; control circuitry configured to operate the array of ultrasound transducer elements between 100 kHz and 1 MHz as a phased array allowing beamforming of emitted ultrasound signals; and a printed circuit board (PCB) substrate configured as a matching layer, onto which a front side of each ultrasound transducer element of the array of ultrasound transducer elements is mounted for transmission of the emitted ultrasound signals through the PCB substrate, further wherein the array of ultrasound transducers are air backed on a back side of each ultrasound transducer element; wherein the control circuitry is on the PCB substrate.

Also described herein are methods of stimulating a subject using the apparatuses described herein, an in particular neurostimulation. For example, described herein are methods of neuromodulation by ultrasound, the method comprising: attaching a phased array ultrasound applicator device to a subject's head, wherein the device comprises an array of ultrasound transducer elements wherein each transducer element has a width and a thickness, and the width is greater than twice the thickness; operating the phased array ultrasound applicator device so that the ultrasound transducer elements oscillate more in a radial mode than in a thickness mode; and beamforming the emitted ultrasound signals from the array of ultrasound transducer elements.

A method of neuromodulation by ultrasound may include: attaching a phased array ultrasound applicator device to a subject's head, wherein the device comprises an array of ultrasound transducer elements wherein each ultrasound transducer element has a width of between about 0.5 mm and 2 mm and a thickness, and wherein the width is greater than twice the thickness, further wherein the spacing between the ultrasound transducer elements of the array of ultrasound elements is between about 0.01 mm and 2 mm, and a printed circuit board (PCB) substrate configured as a matching layer, onto which a front side of each ultrasound transducer element of the array of ultrasound transducer elements is mounted for transmission of the emitted ultrasound signals through the PCB substrate; and emitting ultrasound signals through the PCB substrate between 100 kHz and 1 MHz; and beamforming the emitted ultrasound signals from the array of ultrasound transducer elements.

### BRIEF DESCRIPTION OF THE DRAWINGS

FIG. 1 shows one example of a schematic of an ultrasound phased array apparatus as described herein.

FIG. 2 illustrates one example of array elements loaded onto a surface mount technology tray.

FIG. 3 illustrates one example of an array of ultrasound elements (transducer elements) arranged on a circuit board with printed connectors to each, as described herein.

FIGS. 4A and 4B schematically illustrate (not to scale) different examples of phased array element coupled to a circuit board.

FIGS. 5A-5C illustrate variations of phased array elements as described herein.

FIG. 5E shows a schematic of another example of a schematic of a transducer element as described herein, having a pair of electrodes (one on top and one on bottom) attached to a PCB, showing operation primarily in the radial mode (in which the majority of the energy is radiated outwards from the element as partially illustrated by the dashed lines).

FIG. 5E is another example of a transducer element, showing electrodes attached to the oscillating element on the sides, rather than the top/bottom, so that both electrodes may be in easy electrical contact with the PCB and connecting traces thereon.

FIG. 5F is another example of a transducer element similar to that shown in FIG. 5F, having a cylindrical (rather than cuboid) shape.

FIG. 6 is a schematic illustration through a phased array element coupled to a circuit board that is configured as a matching layer.

FIG. 7 is a schematic illustration through another example of a phased array element coupled to a circuit board.

FIG. 8 is a schematic illustration through two phased array elements coupled to a circuit board, as described herein.

FIG. 9 is a schematic of a section through three phased array elements coupled to a circuit board.

FIG. 10 illustrates a plurality of phased array elements coupled to a circuit board.

FIG. 11 is a schematic section through three phased array elements coupled to a circuit board.

FIG. 12 is a schematic showing a plurality of phased array elements coupled to a circuit board.

FIG. 13 is another example of a schematic of a plurality of phased array elements coupled to a circuit board.

FIG. 14 is another example of a schematic of three phased array elements coupled to a circuit board.

FIG. 15 is a schematic of the components of one example of an ultrasound array.

FIG. 16A shows one variation of an ultrasound phased array applicator configured as a neurostimulation applicator applied to a subject's head.

FIG. 16B shows another variation of an ultrasound phased array applicator having two separate arrays in two separate housings, which may share a common controller, power source, etc., or may each include their own controller and/or power source. The component ultrasound phased array applicators may be physically or wirelessly connected, and may coordinate the applied ultrasound energy.

FIG. 16C shows another variation of an ultrasound phased array applicator device that is worn on a subject's head. The device may operate in communication with a user-held device (e.g., smartphone, pad, laptop, etc.) wirelessly, or it may be configured to operate autonomously.

FIG. 17 diagrammatically illustrates the operation of an ultrasound phased array applicator to adjust the wave front (e.g., phase angle) based on a second set of ultrasound transducers that may detect structures such as bone (e.g., bone thickness) and modify the applied ultrasound energy accordingly.

FIG. 18 shown an example of a plurality of ultrasound transducer devices that may be positioned (e.g., against a PCB substrate) at different angles to help in steering/focusing the ultrasound energy delivered from the ultrasound phased array applicators.

FIG. 19 is an example of a portion of an ultrasound phased array applicator in which timing delays (which may be used for beamforming/steering the emitted ultrasound signals) are built into the apparatus (e.g., between the substrate and each ultrasound transducer).

FIG. 20 is a schematic for regulating the applied energy for delivering ultrasound from the ultrasound phased array applicators described herein. A circuit configured as shown in FIG. 20 may be included as part of any of the ultrasound phased array applicators described herein.

FIG. 21 is an example of a portion of an ultrasound phased array applicator including an imaging sensor that may help target or direct the energy applied by the ultrasound phased array applicators described herein.

FIG. 22 schematically illustrates one method for forming an ultrasound phased array applicator as described herein.

FIG. 23A schematically illustrates one example of a system including an ultrasound phased array applicator as described herein.

FIG. 23B schematically illustrates an example of an ultrasound phased array applicator device as described herein.

FIG. 24 shows one example of a front of a PCB including a 100 element array (10×10) formed as described herein.

FIG. 25 illustrates an 80 Watts, 0.5 MHz switching regulator on top of the array PCB shown in FIG. 24.

FIG. 26 shows one example of a waveform emitted by an apparatus constructed with the PCB shown in FIG. 24; the waveform is emitted through the PCB (which acts as a matching layer).

FIG. 27 is another example of an ultrasound stimulation from an apparatus such as the one described in FIG. 26, operating primarily in a radial mode, and transmitting through the PCB.

### DETAILED DESCRIPTION

In general, described herein are ultrasound phased array apparatuses (including systems and devices) and methods for making and using them. These apparatuses may be thin, and lightweight, so that they may be worn on a subject's head or other body region, comfortably and in acoustic communication with the body so as to deliver ultrasound, e.g., for stimulation of tissue, and particularly for neurostimulation. As a specific example, describe herein are neurostimulation apparatuses that are configured as phased array ultrasound applicators, which are wearable and lightweight, and which may dynamically apply energy to the body. Although wearable neuromodulation devices are described herein, these apparatuses, including ultrasound phased array applicator apparatuses, methods of making the apparatuses, and methods of operating a phased array ultrasound apparatus may be used on other portion of the body, or even for non-biological uses, including generally for any application in which it may be beneficial to apply ultrasound energy from a thin and lightweight phased array apparatus.

The ultrasound phased array applicator apparatuses described herein may include an array of ultrasound transducer elements that are arranged on a PCB. The PCB may be rigid or flexible (e.g., any known printed circuit board/substrate material may be used, including a substrate such as a Kapton, e.g., a polyimide film, and/or vinyl, e.g., coated vinyl, polyvinyl chloride or related polymer). The ultrasound transducer elements may be configured so that they operate primarily in a radial mode of transmission, rather than primarily in a longitudinal, thickness, or other mode. For example, the transducer elements may each have a width and a thickness such that the width is greater than twice the thickness. Thus, the transducer elements may be configured to be driven with a drive signal having a primary frequency that is at or near the resonance for radial mode operation.

In general, these apparatuses are particularly well adapted for delivering energy, using frequencies of between about 20 kHz and about 2 MHz (e.g., between about 20 kHz and about 1.5 MHz, between about 50 kHz and about 1.5 MHz, and particularly between about 100 kHz and 1 MHz). Thus the sizes and configuration (including spacing and arrangement on the substrate, e.g., PCB) of the ultrasound transducer elements may be adapted specifically for operation over this range of ultrasound frequencies, which may refer to the primary frequency range (e.g., greater than 80%, greater than 85%, greater than 90%) of the ultrasound energy applied. In general, ultrasound or ultrasonic radiation may refer to mechanical (including acoustic or other terms of pressure) waves in a medium in the general frequency range from about 20 kHz to about 4 GHz or greater. In some contexts, as specified, the ultrasound referenced is within this relatively lower frequency target range of 20 kHz to 2 MHz, and particularly 100 kHz to 1 MHz.

As used herein, an effective amount of ultrasound may refer to the amount of ultrasound applied as sufficient to elicit one or more desired effects, or achieve one or more therapeutically effective results, including neurostimulation, or the like. Similarly, a therapeutic effect or therapeutically desirable effect may refer to a change in a subject (or region of a subject) being treated such that the subject exhibits the desired effect, in the manner desired, e.g., neurostimulation occurs, a cognitive effect occurs in the subject, the subject reports, and/or feels the effect, etc.

As will be described in greater detail below, the arrays of ultrasound transducer elements are generally secured to a printed circuit board. The thin and lightweight sizes of the array may benefit by arranging the apparatus so that control circuitry is also attached to the PCB, and thus connections may be made on the PCB without requiring additional cabling. The control circuitry may be generally configured to operate the array of ultrasound transducer elements as a phased array, allowing beamforming of emitted ultrasound signals. The control circuitry may include or be connected to a power supply, a processor (including an ASIC, programmable processor), filters, amplifiers, and any other circuitry element.

The printed circuit board may be configured so that the ultrasound signal is transmitted through the PCB. Thus, in some variations the PCB is configured as a matching layer between the ultrasound transducer and the body (e.g., the skin to which the apparatus is applied). This is described in greater detail below.

Although the transducer elements described herein are usually illustrated in the figures as cuboid, it may be a cube, a cylinder, a prism, a pyramid, etc. The transducer elements typically include two electrodes, which may be located on opposite sides of the transducer. The dimensions of the transducer element may generally be configured as phased arrays having a closely-packed arrangement (e.g., separated by less than 2 mm, less than 1 mm, less than 0.5 mm, less than 0.2 mm, less than 0.1 mm). However, larger spacing (which may increase the side lobes) may be desirable in some variations (e.g., >2 mm spacing). The transducer elements may be air backed, meaning that one side of the transducer element (e.g., the side opposite to the PCB) is not connected or contacting on the opposite side, and is therefore unsupported on the side opposite the PCB. Air backing (so that the back of the ultrasound transducer is facing an air backed cavity) may be particularly beneficial to prevent or eliminate loss in energy from the back, and is achievable because the electrical contacts forming end regions of the transducer (e.g., on the ends of a piezo material) may be in direct contact with electrical traces on the PCB.

The phased array applicators described herein may be operated to stimulation by the application of impulses (e.g., fewer than 10 cycles) or with multiple cycles (e.g., 10 or more cycles). The range of frequencies used in many of the devices described here operate primarily within a relatively low frequency range (e.g., 20 kHz up to 2 MHz, or in some variations 100 kHz up to 0.8 MHz) that is different from the range used by many commercial imaging ultrasound devices (which typically operate between 2 MHz to 15 MHz for improved resolution). The aperture size of the apparatuses described herein may be optimized for placement against the skin in a wearable device, with transducers having a width (which may also be referred to herein as diameter) that is generally between about 0.5 and 2 mm. For example, the width may be about or less than about 1 wavelength (e.g., less than about ½ wavelength, etc.) of the dominant frequency. The thickness may be less than about 25% of the wavelength (e.g., 20% or less, 15% or less, 10% or less). The transducers may have a width to thickness ratio that is between about 2 and 10 (e.g., the width is greater than 2× the thickness, e.g., between about 2× and 10× the thickness, etc.). The thickness may refer to just the crystal or other vibrating portion, e.g., excluding the electrodes; in some variations the thickness of the electrodes may be included (and in some cases, may be relatively small, contributing little to the thickness).

Although operation of the transducers in the apparatus primarily in the radial mode as described herein may result in somewhat of a loss of efficiency, compared to thickness mode, operating primarily (e.g., >50% of the transmitted ultrasound energy) in the radial mode may allow the devices to be very thin, particularly as compared to devices operating primarily in the thickness mode (which may have a thickness many times the width, e.g., 2×, 3×, 4×, 5×, etc.). Further, in some variations the sizes and orientation of the transducers may be configured to offset the loss of efficiency. For example, sizing of the transducer as a cubic transducer (e.g., having a width that is approximately 1 wavelength with a thickness that is approximate ½ wavelength) may be operated at higher efficiency but still radiate substantially in the radial mode.

As mentioned, the ultrasound phased arrays described herein may be portable, wearable, hand-held, pocket-sized, and/or otherwise capable of being carried and/or moved. These ultrasound phased arrays may be inexpensive, configurable, customizable, disposable, and/or otherwise tailored for consumer use. In some embodiments, the ultrasound phased arrays described herein may be adapted for neuromodulation. As will be described in more detail below, these ultrasound phased arrays may be used as part of a stimulator, such as a neurostimulator, to alter emotion, increase motivation, decrease pain, track a neural signal, and/or map a neural pathway; these ultrasound phased arrays may be used over-the-counter or as a prescription device.

The ultrasound phased arrays may be used in a wide variety of industrial, medical, and biological applications. In general, any of the apparatuses described herein, and particularly the phased array ultrasound apparatuses described herein that are thin and lightweight enough to be comfortably worn by a subject, may be formed of very small ultrasound transducers of highly uniform shapes and sizes. In making these arrays, it may be particularly beneficial to use an apparatus for placing the ultrasound transducers of the proper dimensions in specified locations on a printed circuit board (PCB) or on a connector (e.g., epoxy, solder, etc.) on the PCB to attach the transducers.

For example, a method for manufacturing small, lightweight, and energy efficient ultrasound phased arrays may use surface mount technology (SMT) or similar approaches. In some embodiments, SMT may be used to manufacture arrays with an area of less than 1 cm² or very large arrays with an area of up to, equal to, or greater than 1 ft².

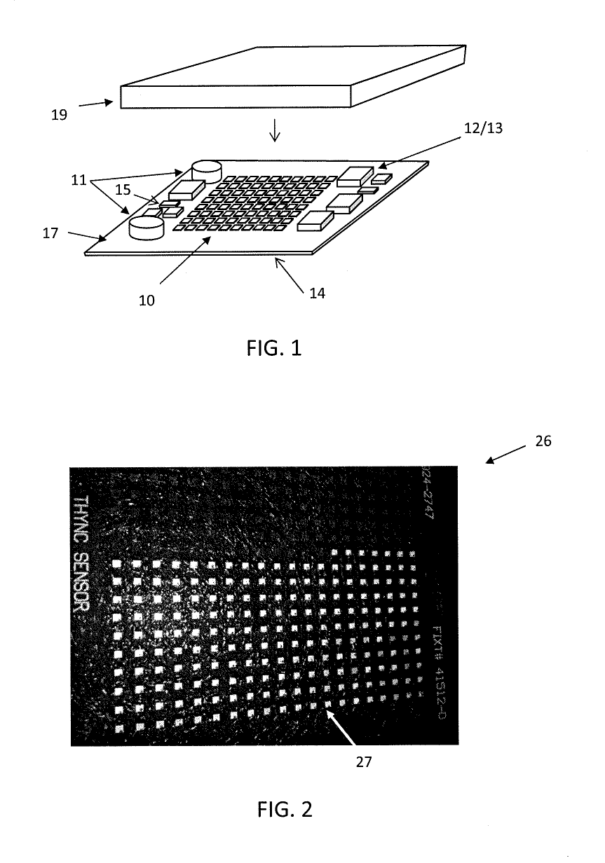

FIG. 1 shows one variation of an ultrasound phased array apparatus including at least one transducer element including a piezoelectric material where each transducer element includes two electrodes coupled to the transducer element, and a circuit board holding an array of transducer elements 10 coupled to the circuit board (PCB 17). In some embodiments, the system may further include a power source (e.g., batteries 11, capacitive power supply, etc.). The system may further include circuitry and leads (e.g., connecting the transducer elements to the circuitry (not shown), an electrical signal generator 12 and an electronic controller 13. The controller may steer and/or focus ultrasound beams. The apparatus may include a housing and/or cover 19, and an adhesive, couplant, or other means of forming an acoustic coupling with the skin may be included on a bottom surface 14. As shown in FIG. 1, the apparatus includes at least one adhesive surface on the underside of the device 14. The ultrasound phased array may further include safety circuitry 15, preventing operation of the device in unsafe conditions and/or limiting the voltages/currents applied to the transducers to limit the application of ultrasound.

The ultrasound phased array in FIG. 1 may be coupled to a machine, tool, instrument, building, user, patient, or any other type of device or human. Coupling may include taping, gluing, soldering, welding, or otherwise adhering the ultrasound phased array to a device, structure, or human. The ultrasound phased array shown in FIG. 1 may be used to stimulate and/or sense or image (e.g., to monitor construction, maintenance, a medical condition, a biological process, and/or any other type of process or application).

Any of the apparatuses described herein may include a circuit board, as shown in FIG. 1, and may be configured to couple to at least one transducer element. The circuit board may be configured to mechanically support and electrically couple elements of the phased array. In some embodiments, the circuit board may include traces either exposed or embedded between the insulating layers of the circuit board to electrically couple elements, including the transducer elements and/or control, power and safety circuitry elements. The traces may be routed to avoid interfering with ultrasound propagation. The circuit board including the at least one transducer element may be configured to deliver ultrasound waves to monitor, assess, and/or image a device, structure, or user. As mentioned above, the PCB may be acoustically matched, and may act as a matching layer. For example, the PCB may include a blend of glass fibers and epoxy, such that the circuit board has optimized acoustic impedance. In some embodiments, the circuit board may be optimized in acoustic impedance and thickness to further function as a matching layer to reduce reflection of the ultrasound waves. Alternatively, the circuit board may include a glass board, ceramic board, or any other type of board suitable for use in an ultrasound phased array.

In some embodiments, the circuit board may function as a ground plane, such that the circuit board may prevent electromagnetic interference where sound propagates through the circuit board. In some embodiments, pogo pins may be implemented into the array when the circuit board functions as a ground plane to establish a contact with the signal side of the transducer element. In some embodiments, the circuit board may include a phase plate, such that the plate creates a time delay and/or phase change for each array element. In some embodiments, the circuit board may include an antenna, for example for Bluetooth, for propagating the sound through the circuit board.

The circuit board may be single sided, double sided, or multi-layered. The circuit board may be rigid, semi-rigid, or flexible, such that the circuit board maintains its original shape or conforms to the shape of the structure to which it is coupled. In some embodiments, a plurality of transducer elements may be coupled to the circuit board, such that each transducer element is less than 1 mm, 2 mm, 3 mm, 4 mm, 5 mm, 6 mm, 7 mm, or 8 mm in radius, preferably less than about 3 mm in radius.

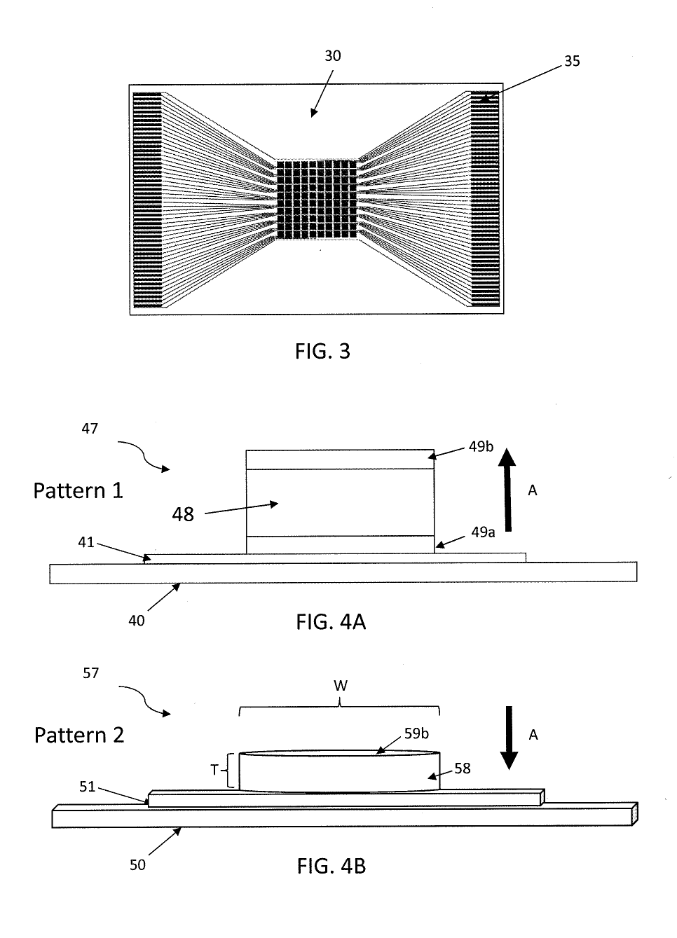

In FIGS. 1 and 3, the circuit board 30 may include a pattern 35 for positioning the ultrasound transducer elements on the circuit board 30 in a predetermined pattern (e.g., a 2-dimensional array). Alternatively, the circuit board 30 may include at least one pad for receiving at least one transducer element.

As shown in FIGS. 1, 4, 5D-5F and 6-14, in some embodiments, the transducer element is coupled to the circuit board or a pad coupled to the circuit board, as shown in FIGS. 4A and 4B. The components may be coupled by reflow soldering, for example with a solder paste 41, 51, as shown in FIGS. 4A and 4B. The solder paste functions to mechanically and electrically couple the components, for example the transducer element, to the circuit board and/or pad, as shown in FIG. 3. In some embodiments, once the components, for example transducer elements, are positioned on the circuit board (e.g., using a robotic pick and place device, e.g., an SMT apparatus), the PCB with placed transducer element(s) may be heated (e.g., using a tube oven, or similar device), to melt the solder paste into an alloy film. In some embodiments, the film may be very thin, with a starting (before heating) thickness significantly less than 250 microns, and heating leading to further reduction in thickness, to achieve high efficiency ultrasound transmission. The solder paste may include tin, lead, bismuth, indium, or any other similar alloy. In some embodiments, the solder paste may include a low melting temperature, for example to be compatible with SMT and the low melting temperature of piezoceramics. For example, the solder paste may include a melting temperature between 50° C.-100° C., 100° C.-150° C., 150° C.-200° C., 200° C.-250° C., preferably less than 180° C. The solder paste once melted may form a thin film between 0.1 mm and 0.4 mm thick, preferably about 0.2 mm thick. In some embodiments, the reflowed solder paste may conduct ultrasound with very little loss, in particular at the frequency ranges of interest herein, e.g., between 0.1 MHz and 1 MHz, or less than about 0.5 MHz. In some embodiments, bubbles in the reflowed solder may be removed by vapor phase reflow and/or by maintaining a low temperature for the reflow.

Alternatively, the components may be coupled with solder flux applied to the PCB via a nozzle before positioning of the components, for example transducer elements, on the circuit board. “No clean” flux may be used. The “no clean” flux may solidify after the heat from the reflow process. Alternatively, in some embodiments, coupling may include a glue and/or epoxy. Heavier components may require a glue and/or epoxy for coupling to the circuit board. In some embodiments, the quality of coupling between the array elements, for example transducer elements, to the circuit board may be inspected optically or by one-dimensional or two-dimensional x-ray.

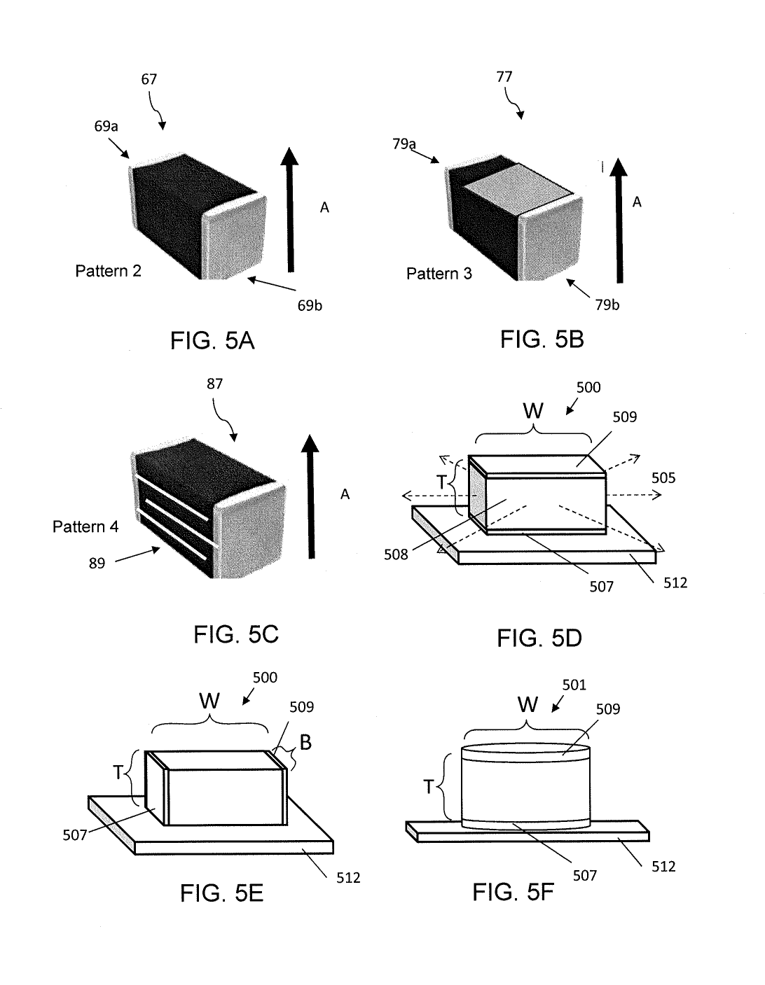

FIGS. 4A-14 show examples of different configurations of transducer elements. In general, a transducer element may include a piezoelectric material and two electrodes coupled to different sides. The transducer element converts electrical energy, e.g., from the circuit board components, into mechanical (vibrational) energy by physical deformation of the piezoelectric material. The transducer may be a radial, lateral, thickness, circumferential, length, longitudinal, shear, or thickness mode transducer element, however as described herein, it may be preferable to provide a transducer that operates primarily in the radial and/or lateral mode, as shown in FIGS. 4A-5F.

As discussed above, the transducer elements described herein for these ultrasound phased arrays may transmit focused ultrasound at frequencies between 20 kHz and 2 MHz (e.g., 100 kHz to 0.8 MHz, etc.), though they may in some cases be configured to operate up to about 5 MHz.

The choice of piezoelectric material largely depends on the acoustic impedance and drive voltage required for driving the transducer element. In some embodiments, the piezoelectric material may include synthetic ceramics, such as barium titanate, lead titanite, lead zirconate titanate (PZT4 or PZT8), potassium niobate, lithium niobate, lithium tantalite, sodium tungstate, zinc oxide, lead metaniobate, or any other type of synthetic ceramic. In some embodiments, PZT4 pillars may be encapsulated in an epoxy matrix to form a piezoelectric material. Alternatively, the piezoelectric material may include synthetic crystals, such as gallium orthophosphate and langasite. In some embodiments, the piezoelectric material may include natural occurring crystals, such as quartz, berlinite, sucrose, Rochelle salt, topaz, and tourmaline-group minerals.

In some embodiments, as shown in FIGS. 4A, 4B and 5A-5F, the two electrodes of the transducer element(s), and the transducer elements themselves, may have a variety of configurations. For example, the material that vibrates in the transducer (e.g., peizo or any other competent material) may be cuboid, cubic, cylindrical, etc. FIG. 4A shows a cuboid material in which the width is greater than twice the height (note that these figures, unless specified otherwise, are not to scale). The dimensions may depend on the wavelength(s) to be delivered. The configurations of the electrodes will be discussed in further detail below. The signal generator may include a driver (which may also be mounted to the PCB). The electronic signal generator (signal generator circuitry) may excite the ultrasound phased array transducer elements. The embedded or exposed traces on the circuit board (PCB) forming the substrate for the array may conduct electrical signals from the driver to the array elements. The driver may deliver square waves, sinusoidal waves, saw tooth (e.g., triangular) waves, etc., including combinations of these, to drive the array; these signals may be timed to provide 2D steering of the array. Alternatively or additionally, the driver may be or may include a switching regulator.

Any of these apparatuses may include a safety circuit. The safety circuit may include a temperature sensor, shut down circuitry (for operating a shut-down mode), and/or may include circuitry for detecting and responding to one or more of: maximum time, average current sensing, average voltage sensing, continuous wave operation, etc. Any type of safety sensing mechanism may be included.

Any appropriate power source may be included. Returning to FIG. 1, the power source 11 may include a disposable or rechargeable battery, capacitive power source, solar panel, external power source, and/or an inductive charging source, such as wireless charging panel. A battery may further function as a backing layer to optimize array performance, such that the battery may reduce excessive vibration of the piezoelectric material in the transducer element.

For any of the wearable devices described herein, the apparatus may include a mount for connecting the apparatus to a subject, including acoustically coupling the apparatus. For example, the mount may be an adhesive, and/or a strap etc. As shown in FIG. 1, the ultrasound phased array may include at least one adhesive surface 14. In some embodiments, the array may include an adhesive surface 14 on two opposing sides of the array. In some embodiments, the adhesive surface may include glues, cyanoacrylates, toughened acrylics, epoxies, polyurethanes, silicones, phenolics, polyimides, plastisols, polyvinyl acetate, pressure-sensitive adhesives, or any other type of suitable adhesive. In some examples, the apparatus may be integrated into a garment (e.g., headband, hat, shirt, pants, sleeve, etc.) that may be used by itself or in addition to another material and/or structure, to hold the apparatus in position against the subject's body (e.g., skin).

As mentioned above, when fabricating the arrays described herein, it may be particularly useful to use a surface mount technology. FIG. 2 illustrates array elements loaded onto a surface mount technology tray 26. As shown in FIG. 2, the components 27 of the array, for example the at least one transducer, may be “picked” from a surface mount technology (SMT) ready tray 26 and “placed” on the circuit board using SMT. Alternatively, an SMT component shooter may be used to position transducer elements with an air jet onto the circuit board. SMT may be used to manufacture smaller, thinner, and less expansive arrays than previously possible. In some embodiments, using SMT enables non-uniform spacing between elements, which is beneficial for reduction of grating side-lobes (i.e. phenomena of sound energy spreading out from a transducer at angles other than the primary path). Further, using SMT enables positioning of components at precise locations for increased beam performance. Array elements may be positioned and/or angled on the array to enhance steering and focusing of array components. Requirements for phase accuracy may be reduced by positioning components in the best location on the array for phase quantization. SMT further enables the use of lateral and radial mode resonance array components. Additionally, using SMT eliminates manual connections and cables in favor of direct connection between array components and driving electronics through routing traces in the circuit board, significantly reducing thickness and weight of the system and simplifying the construction process. The apparatuses formed (e.g., using SMT) as described herein may also eliminate the need for mechanical lenses, such that the array may directly contact the device, structure, and/or user, particularly when the apparatus is oriented so that the back of the PCB may be placed in contact with the subject (though an additional intervening layer or coating may be applied to the back of the PCB); the PCB may act as a (or part of a) matching layer for transferring ultrasound energy from the transducer array to the patient's body.

SMT may be used to include built-in phase delays into the array. The delay may be due to active components (e.g., electronic components, e.g., circuitry) or passive designs (e.g., trace lengths, positions of the transducer elements, etc.). For example, SMT may be used to configure the apparatuses to produce 180 degree phase delays, such that the polarity of array components may be switched.

An example of a PCB formed as described herein is shown in FIG. 3. FIG. 3 illustrates a printed circuit board 30, as described above, in accordance with a preferred embodiment. In some embodiments, the circuit board 30 may include a printed pattern 35 of traces or connectors connecting to the transducers (and particularly the electrodes on the transducer(s) and/or other active circuitry elements, such as the controller, power supply, etc.). The pattern may be uniform or non-uniform. In some embodiments, the pattern may be utilized to create a desirable pattern of transducer element location, for example to reduce grating side-lobes, as described above. Manual or automated optical inspection may be used to position and/or align transducer elements and other array components on the pattern 35 on the circuit board. The pattern 35 may reduce or eliminate discrete phase delays required to achieve steering and focusing.

FIGS. 4A and 4B illustrate one example of a phased array element 47, 57 coupled to a circuit board 40, 50. In FIG. 4A, the vibrational component 48 is a cuboid shape, while in FIG. 4B, it is a cylindrical shape 58. A pair of electrodes is attached; in FIG. 4A two electrodes couple to the vibrational component of the transducer element (e.g., the piezoelectric material) and may in turn be coupled directly or indirectly to the PCB 40, 50. Application of a voltage to the electrodes induces expansion and contraction of the transducer element leading to vibration and sound. The electrodes may be formed of copper, graphite, carbon, titanium, brass, silver, platinum, palladium, mixed metal oxide, or any other suitable metal or substance, including mixtures of these.

Electrodes may be positioned in any appropriate pattern. For example, the electrodes on each piezo of the transducer may be coupled to opposing sides of the transducer element and/or piezoelectric material, as shown in pattern 1 in FIG. 4A. The transducer element may be a lateral mode transducer, or a radial mode transducer. In pattern 1, a first electrode 49a is shown coupled to the piezoelectric material 48 and to a pad 41 or circuit board 40 while the second electrode 49b is coupled to the transducer element 48. The second electrode 49b may be a ground electrode. In some variations, the second electrode may be placed in electrical contact with the user when the apparatus is in acoustic contact with the user, and this contact may serve to ground (e.g., complete the circuit) for applying energy to the transducer element; the second electrode 49b may interface, contact, or otherwise interact with the device, structure, or user. In some embodiments, pattern 1 may be appropriate for neuromodulation uses. Alternatively, the second electrode 49b may be connected (e.g., via a trace or wire) to a ground (e.g. common ground) or reference for the transducer elements. The electrodes may be wrap-around electrodes, and may be on the same, or different (including opposite, as shown in FIGS. 4A and 5A-5F) sides of the piezoelectric material.

In some embodiments (e.g., particularly for lateral mode resonance), the width of the element is approximately the sound velocity of the transducer material divided by two times the frequency of the array element (e.g., width=c/2f). For example, in FIG. 4A the width of the piezoelectric material of the transducer element 47 may be approximately 4 mm for a 0.5 MHz resonance. The thickness may be set by the mode (e.g., resonance) used to drive the transducer and can be as thin as 0.1 mm.

In FIG. 4A or FIG. 4B, the transducers may be configured to operate in a radial mode (and/or lateral resonance) which may be preferred for use in thin arrays where the aspect ratio of width to thickness is between 1 to 20 (or any sub-range within 1-20, such as 2-10, e.g., 1, 2, 3, 4 or 5 to 20, 19, 18, 17, 16, 15, 14, 13, 12, 11, 10, 9, or 8); traditional array elements may have an aspect ratio of width to thickness between 0.4 to 0.6. The resonance frequency of the transducer to operate primarily in a radial mode may be influenced by the width and length of the piezoelectric material, facilitating their use in smaller, thinner arrays.

In FIGS. 4A and 4B, the arrangement of the electrodes may include connections to the PCB, which may be defined during fabrication using SMT. SMT may include using a solder resist, solder mask, or solder stop mask slightly taller than the solder thickness around the circuit board pad to form the connections to the transducer element electrodes. For example, a film with a metallic coating may be used to cover multiple array elements, and a conductive compound, such as silver epoxy, may form an electrical contact between the electrodes and the film to serve as ground (and in some variations, a common ground).

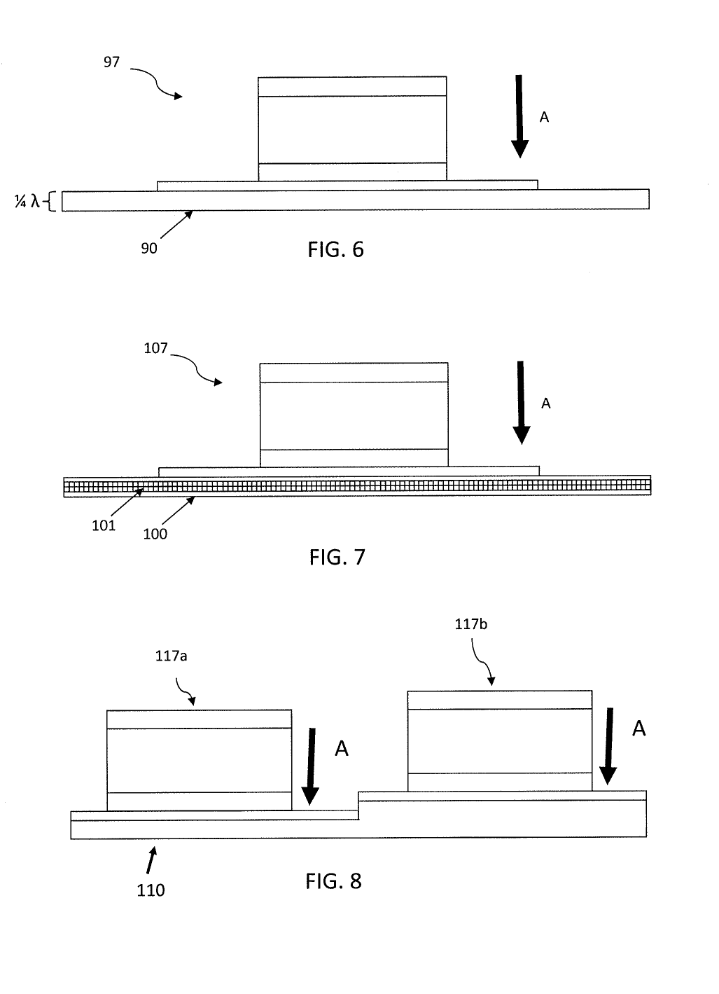

The primary focus of the ultrasound energy may propagate in a direction A away from the circuit board. In FIG. 4A, the direction of propagation is primarily away from the PCB (arrow A pointing up), while in FIG. 4B, the direction of propagation is primarily through the PCB. Thus, as described herein, the ultrasound wave may propagate in direction A towards and through the circuit board, as shown in FIGS. 6-8.

FIGS. 5A through 5C illustrate different variations of phased array elements (showing electrode patterns 2, 3, and 4). Electrode pattern 2, as shown in FIG. 5A, is an end-cap electrode pattern in which the first and second electrodes 69a, 69b (respectively) of the transducer element, are positioned parallel to the direction A of sound propagation towards the device, structure, or user. As shown in FIG. 5A, the ultrasound energy does not (primarily) pass through the first or second electrodes. Depending on the operational mode, the (e.g., lateral) compression of the piezoelectric material in the transducer element 67 due to an electric field from the end cap electrodes 69a, 69b may induce alterations in the thickness of the piezoelectric material to the Poisson ratio of the piezoelectric material. Alternatively, radial mode of array element resonance may be used, such that ultrasound waves move through the electrodes.

In some embodiments, electrode pattern 2 may be more cost-effective than other electrode patterns since existing equipment for SMT resistors and capacitors may be used. Further, during SMT, use of reels for packaging instead of trays, and standard air nozzles for the positioning of the array elements onto the circuit board in pattern 2 increases the speed of manufacturing to about 10 elements per second or 600 elements per minute. In some embodiments, electrodes in the end-cap electrode configuration, pattern 2, may self-align during coupling to the circuit board.

As shown in pattern 3 in FIG. 5B, the electrodes may wrap around the transducer element 77, such that the second electrode 79b, for example the ground electrode, may be disposed in the same plane as the first electrode 79a and both electrodes may contact the circuit board. This may tolerate/reduce drifting of the array element during manufacturing and reduce or eliminate the need for a common ground metal film. Ultrasound energy may propagate in a direction A away from the circuit board, as shown in FIG. 5B, radially outward (not shown).

In some embodiments, as shown in pattern 4 in FIG. 5C, the electrodes 89 may be distributed in multiple layers through the transducer element 87 including the piezoelectric material. In pattern 4, multiple plates of the piezoelectric material may be stacked and mechanically coupled to each other, with electrodes 89 from each of the plates connected in parallel. In such a multi-layered design, the electrical and ground signals may be routed through separate layers in the transducer element 87. This is similar to the multi-layer ceramic capacitor in construction, except the ceramic is replaced by the piezoelectric material. In some embodiments, the polarity of the piezoelectric material may alternate between each of the layers, such that the particle displacements are of the same direction along the stack. In some embodiments, a multi-layered electrode pattern may increase particle displacement and/or lower the drive voltage required. Pattern 4, as shown in FIG. 5C, may enable multiple piezoelectric materials to be packaged within one transducer element 87.

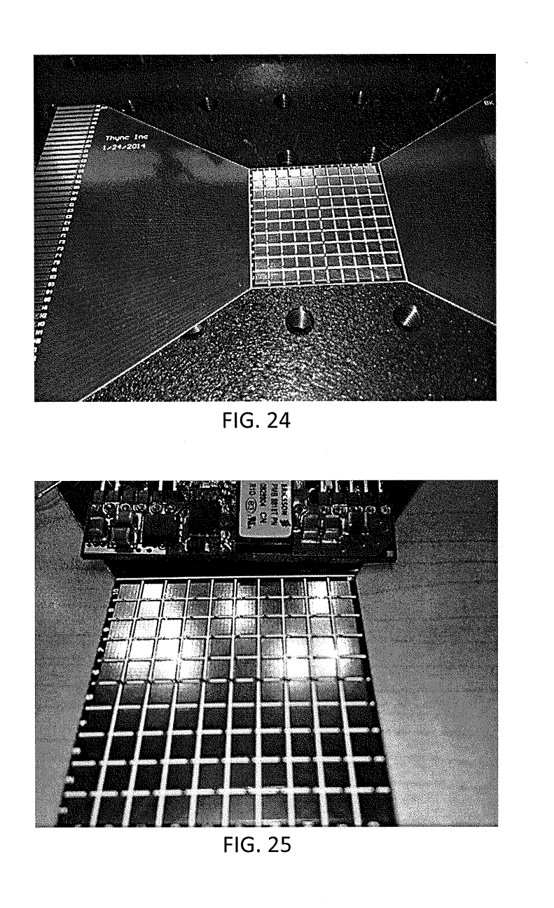

FIG. 5D illustrates one variation of a transducer element, showing the orientation of the element, particularly when operating in a radial mode. In this example, a pair of electrodes 509, 507 are positioned on opposite sides of the piezoelectric material 508. The transducer has a thickness, T, that is less than the width, W, and is mounted to the substrate 512 on the width. The primary direction of ultrasound energy may be directed through the substrate, which may be a PCB, as described above. A backing layer opposite from the substrate may be present or absent (e.g., air backed). This transducer may be arranged in an array (e.g., an x by y array, where x greater than or equal to 1, and y is greater than or equal to 2, e.g., x>10 and y>10, x>20, y>20, etc.). For example, 2D arrays of between 1 and 1000 transducer elements (e.g., 1, 2, 3, 4, 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 300, 400, 500, etc.) by between 2 and 100 transducer elements (e.g., 1, 2, 3, 4, 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 300, 400, 500, etc.) may be used. For example a 10×10 (100 element) array is shown in FIGS. 24-25.

FIGS. 5E and 5F illustrate alternative variations of transducer elements similar to the variation shown in FIG. 5D. In FIG. 5E, the electrodes (ground 509 and active 507 electrodes) are positioned laterally on the transducer, so that they both contact the substrate 512. In this example, the thickness T is much smaller than the width W and breadth B (which may be the same length, or approximately the same length, e.g., W=B). Similarly, in FIG. 5F, the element is a cylindrical element, and includes width (diameter) W that is more than twice the thickness T. The electrodes may be located anywhere on the vibrational (e.g., piezoelectric) material, including on opposite sides (as shown in FIG. 5F) or on the same side (e.g., bottom surface 507).

FIG. 6 illustrates a phased array element 97 coupled to a PCB 90. The circuit board 90 may function as a quarter wavelength (¼λ) matching layer, as shown in FIG. 6. The matching layer provides the interface between the raw transducer element 97 and the user, device, or structure and minimizes the acoustic impedance differences between the transducer 97 and the user, device, or structure. This matching layer may consist of one or more layers of materials with acoustic impedances that are intermediate to the acoustic impedance of the user, device, or structure and the transducer material. The thickness of each layer may be approximately ¼ wavelength (λ), determined from the center operating frequency of the transducer 97 and speed characteristics of the matching layer. For example, the ratio of the fiber glass to epoxy compound in an FR4 circuit board material may create an acoustic impedance suitable as a matching layer for the optimal transmission of ultrasound from a transducer element to a user. Thus, the thickness of the FR4 circuit board 90 may be made a quarter of the acoustic wavelength (¼λ), as shown in FIG. 6. Operation in the 20 kHz to 2 MHz (e.g., 20 kHz to 0.8 MHz) ranges described herein, as opposed to the higher-frequency operation of traditional (e.g., imaging) ultrasound devices may make this more feasible. Alternatively, any other type of circuit board materials, may be used as a matching layer in the phased array. By transmitting ultrasound through the circuit board, direction A, the efficiency of the transducer may be optimized. In addition, holes may be drilled or etched out at the locations of the ultrasound transducer element and later filled with suitable coupling medium such that there is even greater transmission of ultrasound.

Alternatively, the matching layer may include airgel or micro-balloons compressed with hydraulic pressure and fixed in a silicone or epoxy binder. In some embodiments, the micro-balloons may be compressed under pressure and filled with a low viscosity epoxy binder. Conversely, a passive matching layer on the device, structure, or user's skin may enhance energy coupling, such that the matching layer is ¼ wavelength in thickness and includes glass micro-balloons or an aerogel.

FIG. 7 illustrates an example of a phased array element (transducer element) 107 coupled to a PCB 100. In some variations, such as the example as shown in FIG. 7, a mesh type, or any other type of material, ground plane 101 may be included on the circuit board 100, such that ultrasound propagates with minimal attenuation while maintaining electromagnetic interference shielding function. A ground plane 101 may limit acoustic energy transmission between individual elements and serves as a return path for current from the many different components on the phased array.

In some variations, the transducer elements may be arranged to provide lensing and/or focusing of the energy via the substrate. For example, FIG. 8 illustrates two phased array elements 117a, 117b coupled to a PCB 110. In FIG. 8, the PCB 110 may function as a Fresnel lens, such that a phase delay exists between a first transducer element 117a and a second transducer element 117b. A Fresnel lens type of circuit board 110 may be used in applications requiring fixed steering and focusing of the ultrasound beam(s). In some embodiments, each segment of the Fresnel lens may correspond to a 360° phase offset of the ultrasound energy. The Fresnel lens may function as a four-phase system such that the possible phase shifts would be 0°, 90°, 180°, 270°. Alternatively, the Fresnel lens may function as an eight-phase system such that the possible phase shifts would additionally include 45°, 135°, 225°, 315°. In some embodiments, the possible phase shifts may be any combination of degrees.

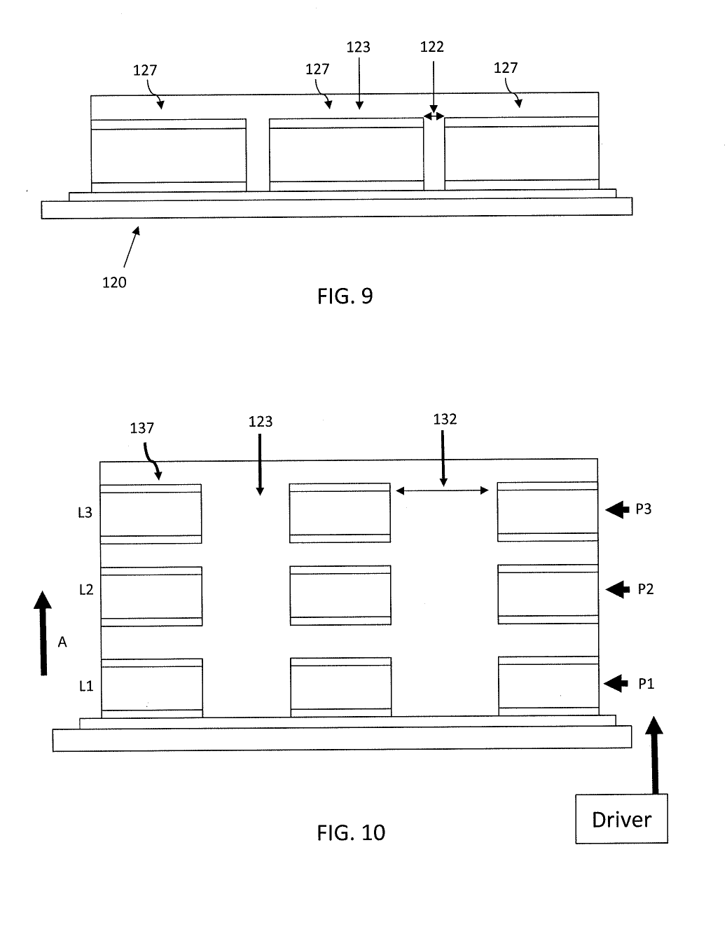

FIG. 9 illustrates three phased array elements 127 coupled to a PCB 120, in which the phased array may have increased stability and water resistance. Gaps 122 between the array components, for example the transducer elements 127, may be filled, packed, and/or saturated with a filler material 123, for example air-filled balloons and/or an elastomer compound such as epoxy, as shown in FIG. 9 and as described above in accordance with FIG. 6. In some embodiments, the gaps 122 between array components may be filled with a low acoustic impedance material to decrease aggregate acoustic impedance of the transducer to better match air. For example, the filler 123 may be glass. The aggregate acoustic impedance of an array may be adjusted by altering the volume ratio of the piezoelectric material to the filler 122. The aggregate impedance of a transducer 127 may be the mean density of the piezoelectric material and the filler 122 between array elements 127. For example, in some embodiments, the aggregate impedance of an array layer may be controlled by using a low-density material between array elements.

In FIG. 10, multiple transducer elements 137 may be stacked and the gaps 132 between the multiple transducer elements filled with a filler material 133 as described above, such that the transducer elements 137 create a transformer effect. In some embodiments, each layer of the stack L1, L2, L3, . . . may include progressively lower acoustic impedance towards the device, structure, or user until the last layer has an acoustic impedance very close to that of air. An example of one field of use of an air-coupled transducer is neuromodulation without physical contact between the phased array and the user. Phase delays, P1, P2, P3, etc. . . . may be added at each layer L1, L2, L3, . . . of the stack. In some embodiments, a drive signal with electrical phase designed to superimpose on the sound wave coming through to a layer may be delivered to each of the layers.

In FIG. 10, the driver signal to each of the transducer layers L1, L2, L3, . . . may be adjusted so that it is in-phase with the ultrasound energy passing through the layer. This superposition effect may increase the ultrasound magnitude. As shown in FIG. 10, adjusting the driver's phase enables constructive superposition along the progressively lower impedance transducers.

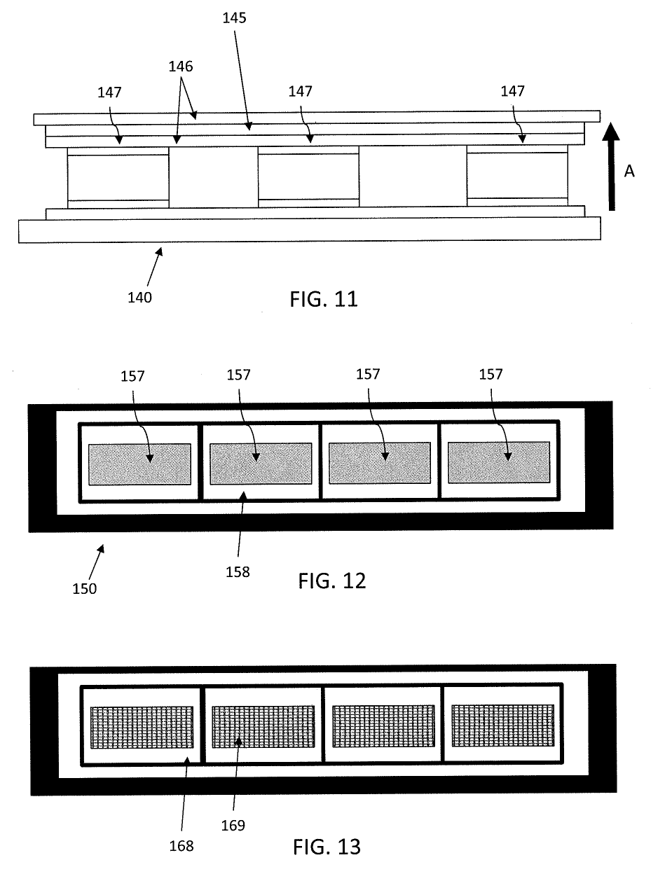

FIG. 11 illustrates three phased array elements 147 coupled to a circuit board 140, in accordance with a preferred embodiment. In some embodiments, it may be desirable to couple the phased array to a device, structure, or user through the array elements 147 and not a circuit board 140, as described above. As shown in FIG. 11, a thin polymer film 146 with metallic coating 145 may be deposited on the face of the circuit board 140 including the components already coupled to the circuit board 140. The solder paste, conductive adhesive, or other coupling material may be applied to the metallic coated layer before positioning the array on the film 146 to achieve electrical connection to the array element. After SMT, the metallic coating 145 may become the common ground to the array element(s) 147. In some embodiments, using the thin polymer film 146 with metallic coating 145 may be useful for arrays including a large number of layers of electrical conductors or in high frequency operation where the attenuation on the circuit board may be problematic.

FIGS. 12 and 13 illustrate a plurality of phased array elements 157 coupled to a circuit board 150, in accordance with a preferred embodiment. In some embodiments; as shown in FIGS. 12 and 13, the array may further include a ground shield 158. The ground shield 158 may be a circular or square toroid, such that the ground shield includes a center aperture for delivering ultrasound while maintaining the electromagnetic interference shielding effect intact. In some embodiments, the wavelength of the electromagnetic wave may be increased relative to the aperture in the ground shield 158. As shown in FIG. 12, the ground shield 158 may include copper or any other conductive or magnetic metal or material. Optionally, as shown in FIG. 13, a fine mesh 169 may mask the opening of the ground shield 168 in cases where higher frequency electromagnetic waves need shielding. In some embodiments, the amount of electromagnetic interference reduction depends on the material used, its thickness, the size of the shielded volume, and the frequency of the fields of interest and the size, shape, and orientation of the apertures in the ground shield 168 relative to an incident electromagnetic field.

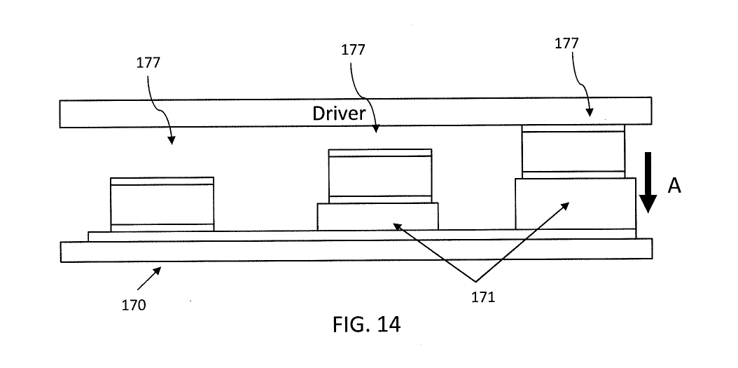

FIG. 14 shows three phased array elements 177 coupled to a circuit board 170. As shown in FIG. 14, using a very thin array element 177, for example a radial or lateral mode transducer, enables the inclusion of propagation delay lines (phase delays) 171 to the array elements 177 to steer the beam without making the array too thick. Adding a 60° phase delay to an element using a polyurethane propagation path in this example results in a total thickness of the module of about 0.55 mm (0.45 mm phase plug plus 0.1 mm element thickness, or any other appropriate thickness for the elements, including very thin elements as described herein). The array illustrated in FIG. 14 may be ideal for wearable applications of an ultrasound array, for example for neuromodulation.

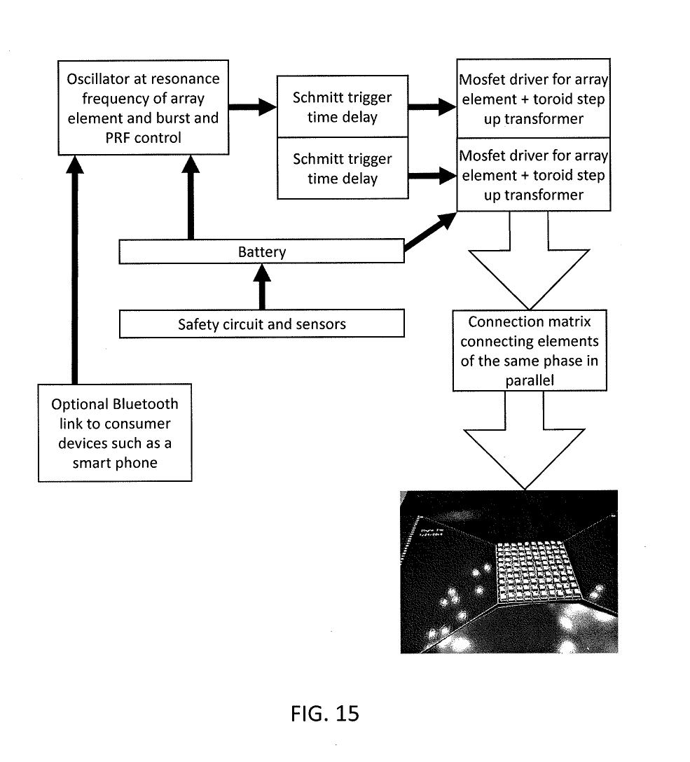

FIG. 15 illustrates a schematic for an ultrasound phase array as described, and FIG. 22 illustrates one method of forming it. In this example, the componentry of an ultrasound array may be sequentially positioned on the substrate (e.g., PCB) by SMT or another mechanism for consumer use. The transducer elements may be formed 1600, and placed (e.g., by SMT) onto the prepared PCB 1610, where they are secured in place (e.g., by soldering, epoxy, etc.) 1620. Either before or afterwards, additional circuitry may be added to the PCB (which may have initially been formed with the traces formed thereon). In some variations, multiple (e.g., stacked) PCBs may be used. For example, an ultrasound array for a neuromodulator, as one example, may include an optional Bluetooth link for connecting to a consumer device such as a laptop, mobile device, or other portable device. In some embodiments, the array may include an oscillator, for example a square type, for inducing a resonance frequency of an array element, burst effects, and pulse repetition frequency. The array may further include a battery and safety circuits and/or sensors, as described above. In some embodiments, the array may include one or more comparator circuits, for example one or more Schmitt trigger time delays. Further, one or more Mosfet drivers and/or toroid transformers may be coupled to the array, such that the Schmitt trigger time delay and battery power electrically feed into the Mosfet drivers and toroid transformers. The above componentry of the same phase may be coupled to the array in parallel. The componentry may be coupled to a circuit board, as shown in FIG. 15.

In FIG. 22 the method of manufacturing an ultrasound array (a phased array) may include the steps of molding a plurality of ceramic elements in a shape for the circuit board, mounting a transducer element comprising two electrodes directly on a circuit board, and coupling the transducer element to the circuit board. Additional circuitry may also be coupled to the PCB, such as the driver and a power source to the circuit board 1630.

Coupling may include reflow soldering the at least one transducer element to the circuit board. A solid plate of ceramic with scribed “break lines” may be reflowed onto a circuit board, then broken into pieces along the scribe line by positioning the circuit board subassembly onto a hemispherical mandrel and applying pressure to the ceramic. The coupling may further include sintering the at least one transducer element to the circuit board. Multiple elements may be interconnected with a “bus” before sintering and/or reflow soldering. The electrode may be deposited and/or fired onto the array after sintering and/or reflow soldering to interconnect the elements, as described above.

Forming or making the phased array ultrasound apparatuses described herein may also include molding a plurality of ceramic elements in a shape to fit together and form the array on the printed circuit board. The coupling may further include coupling at least one of an imaging and therapy element to the circuit board, for example for neuromodulation. As mentioned, the method may also include filling gaps on the circuit board with one of an air-filled balloon and an elastomer. The method may further include coupling an electrical signal generator, such as a driver, to the circuit board. The method further includes coupling a power source to the circuit board.

As mentioned, the ultrasound phased arrays described herein may be used in a wide variety of industrial and non-invasive medical and biological applications. The phased arrays described herein may also be used for invasive application, for example in an implant. The ultrasound phased arrays described herein may be adapted for neuromodulation, and for attachment to the subject's head. However, the ultrasound phased arrays of the present application may also or alternatively be worn on a user's head, arms, legs, chest, back, or any other body part responsive to ultrasound waves. Although in many of the examples described herein the apparatuses are configured so that they do not image tissue (e.g., the ultrasound is primarily for energy delivery, and does not include or need to include using/interpreting the ultrasound for imaging), in some variations these apparatuses may be used or configured for use in imaging instead or in addition to stimulation. For example, the ultrasound phased array of the present application may be used to enhance a state of calmness or relaxation, increase subjective feelings of energy and/or physiological arousal, alter emotion, increase motivation, decrease pain, track a neural signal, and/or map a neural pathway. The ultrasound phased arrays may be used to image neural pathways before, during, and/or after delivery of ultrasound beams. The ultrasound phased arrays described herein may be manufactured to steer and/or focus ultrasound beams to improve neuromodulation. In some embodiments, the ultrasound phased arrays described herein may be used over-the-counter or as a prescription device. As a prescription device, the array may be used to modulate a single neural function, such that when the array is positioned on the skin of the user in the correct location, the specific neural pathway is modified. As an over-the-counter device, the device may be user-actuated (i.e. self-controlled).

In general, the effects induced by ultrasound may be associated with a neural stimulation event. For example, the neural stimulation event may include a user using a hearing aid, glucose monitor, communication device, such as a mobile device, learning device, such as electronic books, or listening to music or a lecture, or any other type of event or device that results in neural stimulation. In some embodiments, ultrasound may emphasize, de-emphasize, and/or focus information. Alternatively, the array may be used to achieve a de-focusing effect, such as a general enhancement or suppression of neural activity. Thus, one use of these apparatuses and methods may be the synchronized pulsing of relevant information to be attended to with neurostimulation via the apparatuses and methods described herein. One such example could be the display and auditory input of a new foreign vocabulary word along with the native tongue equivalent every one second, with an ultrasound stimulus inducing enhanced focus delivered every second. In between these words, there may be no stimulation, stimulation to enhance relaxation, continuous stimulation to cause inattention to any external distracting stimuli, stimulation to intercept unexpected distracting stimuli, and/or target a separate language memory area for consolidation. This is made possible by the fast processing of modern computing and electronics, and that ultrasound can travel at speeds of greater than 1400 m/s in biological tissue. These speeds are sufficient to reach target brain areas and intercept the incoming neural transmission from distracting stimuli as sensory processing in general can take 100 ms or more to reach many higher brain regions.

In general, application of ultrasound to neural activities may produce a net effect on a user's motivation. Effects on motivation were first observed as a result of trans-cranial direct current stimulation and may be relevant to ultrasound. In some embodiments, a phased array may provide feedback to the user of one or more physiological states, such as blood glucose level. For example, typically a user who noticed an increase in blood sugar knows that action should be taken, such as starting to exercise to burn the extra sugar in the blood. However, compliance has been low in the motivation to take action. Thus, neural modification using ultrasound may increase the motivation of the user to exercise to burn extra blood sugar.

Another example of a class of cognitive effects that may be evoked includes those associated with relaxation and a calm mental state, for example: a state of calm, including states of calm that can be rapidly induced (i.e. within about 5 minutes of starting a TES session); a care-free state of mind; a mental state free of worry; induction of sleep; a slowing of the passage of time; enhanced physiological, emotional, or and/or muscular relaxation; enhanced concentration; inhibition of distractions; increased cognitive and/or sensory clarity; a dissociated state; a state akin to mild intoxication by a psychoactive compound (i.e. alcohol); a state akin to mild euphoria induced by a psychoactive compound (i.e. a morphine); the induction of a state of mind described as relaxed and pleasurable; enhanced enjoyment of auditory and visual experiences (i.e. multimedia); reduced physiological arousal; increased capacity to handle emotional or other stressors; a reduction in psychophysiological arousal as associated with changes in the activity of the hypothalamic-pituitary-adrenal axis (HPA axis) generally associated with a reduction in biomarkers of stress, anxiety, and mental dysfunction; anxiolysis; a state of high mental clarity; enhanced physical performance; promotion of resilience to the deleterious consequences of stress; a physical sensation of relaxation in the periphery (i.e. arms and/or legs); a physical sensation of being able to hear your heart beating, and the like. This class of cognitive effects may be referred to collectively as “a calm or relaxed mental state”. The calm effect may be achieved by stimulation of appropriate brain regions, such as the region over or near the temples, mastoid, and regions between the temple and mastoid.

Another example of the class of cognitive effects that may be evoked using the neurostimulators described herein generally results in the subject experiencing an increased mental focus and may include: enhanced focus and attention; enhanced alertness; increased focus and/or attention; enhanced wakefulness; increased subjective feeling of energy; increased objective (i.e. physiological) energy levels; higher levels of motivation (e.g. to work, exercise, complete chores, etc.); increased energy (e.g., physiological arousal, increased subjective feelings of energy); and a physical sensation of warmth in the chest. This class of cognitive effects may be referred to collectively as enhancing (or enhanced) attention, alertness, or mental focus. For example, neurostimulation may be provided by attaching the apparatus at or near the forehead and/or in regions. For example, to evoke a focused attention effect, the apparatus may be applied to the activate associated neural region(s). The default mode network (a distributed functional network in the cerebral cortex) exhibits reduced activity during sustained attention and increased activity during mind-wandering and daydreaming. The right anterior insula and frontal operculum (along the inferior frontal gyrus) have been identified in functional magnetic resonance imaging (fMRI) studies as brain regions activated during sustained attention. The placement of electrodes in this configuration may increase the activity of areas near the right inferior frontal gyrus (including the right insula) and reduce activity in the default mode network, but other brain regions may be activated, inhibited, or modulated in at least some instances. A first electrode may be placed over the right inferior frontal gyrus near position F8 on the 10/20 standard and a second electrode near position AFz (e.g., using 10/20 electrode locations).

In some embodiments, ultrasound may be used to control, reduce, or otherwise suppress pain in the central nervous system. Alternatively, pain may be controlled and/or suppressed in the peripheral nervous system, for example by positioning the phased array on the back, legs, arms, or any other body location.

In some embodiments, ultrasound may be steered to follow the propagation of a neural impulse using a phased array system. Ultrasound propagation speed is faster than neural propagation.

Thus, ultrasound may be used to track propagation of an impulse in real time and stimulate at multiple spatial points along the pathway. If the timing of stimulation is controlled precisely, the effect of tracking will be similar to a “parametric amplifier” or “difference frequency generation,” in which a huge gain may be achieved by pumping at several points along the signal pathway synergistically and in some variations, in-phase with the propagation of the signal. By following the spatial location of specific neural pathways, especially in a temporally coherent manner, the effects of ultrasonic neuromodulation can be made more specific than the spatial resolution (beam width) of the ultrasound focus itself allows. This is because the non-target regions would receive modulation waveforms for a small fraction of the time, while the specific pathway is constantly targeted by the waveform. This scanning method further reduced dosage levels required for neural modulation of the desired pathway and any potential undesired or unnecessary thermal or mechanical effects of target and non-target paths and regions.

Thus, described herein are methods to make ultrasound more precise in the selection of neural function by a pattern of steering that follows the impulse propagation, so as to interact at more than one point with the neural event in synchrony with the impulse propagation. One potential use of such a method is that one may get strong impulses from the amygdala in a fear reaction that can be detected and shunted in anxiety, PTSD, and depression scenarios to prevent maladaptive activity or effects in downstream neural structures.

The speed of impulses along specific pathways can be estimated from DTI of the white matter tracts and diffusion coefficients, clinical and animal studies testing speed of conduction, or the use of test activations of focal brain areas using ultrasound and measuring the subsequent latency of activation in connected brain regions of interest.

A simpler, more static use of stimulation along a pathway is for the modulation of pathways between two specific brain regions in a specific direction. Suppose brain region A were connected to brain regions B and C, and both B and C connected back to A. Simply stimulating brain region A would change the connections strengths of all the pathways in both directions. Stimulation of A and B simultaneously would result in more specific changes, but for both directions of the pathway. Stimulation along a pathway however opens the potential for enhancement or depression of connections in a specific direction between two brain regions.

These stimulation schemes may be combined along with environmental therapies where the subject is exposed to specific visual, auditory, and other sensory stimuli or asked to perform specific tasks that engage the brain regions of interest, further enhancing the effects of brain pathway stimulation.

Similar scanning can be used to scan along a small volume of brain in order to further correct for aberrations caused by skull bone, to adjust for individual anatomical differences, or identify specific target regions of functional interest (epileptic foci, functional sub regions of the brain corresponding to a specific body part or sensory perception, etc.), and aided by signals observed in brain recordings (i.e. EEG), physiological readouts (HRV, blood pressure, GSR), or conscious user feedback. Separate electrical paths for ground could be used to reduce noise in signals. Alternatively, the electrical sensing and ultrasound power-return ground could be the same electrical pathway, with the two functions performed at different times to reduce noise. The functions could be alternated in a strobing manner for semi-continuous monitoring and stimulation, or could be done such that electrical sensing is the default mode until a specific triggering signal is detected, and ultrasound stimulation is initiated.

In operation, the phased array ultrasound apparatuses described herein may be steered, including beam formed, to specifically direct/guide the ultrasound energy relative to the body being stimulated. For example, enhanced steering and/or focusing may be achieved by built in phase delays, polarity reversal, array element location, and/or delay inherent in array elements. A physical time delay element may be added to each array element. Examples of physical (e.g., passive) time delay elements include a propagation path, a phase plate, and/or a variation in thickness of the circuit board. Polarity of an array element may be reversed by flipping the array, which results in an 180° phase shift. Element location may impact steering and focusing, such that an array element may be positioned at a physical location that corresponds to a phase requirement for steering and/or focusing. In some embodiments, the physical location of an array element is defined by the pattern on the circuit board. For delay inherent in array elements, for example, the width to thickness ratio of an element or the number of layers stacked to form each array element may alter steering and/or focusing.

FIG. 16A illustrates one example of a wearable phased array, in accordance with a preferred embodiment. The phased array 1641, as shown in FIG. 16A, may be positioned on a forehead of a user, such that the ultrasound beams are directed to a brain region of a user for neuromodulation. Alternatively, the phased array may be positioned on any body portion of a user, for example to modify the peripheral nervous system of the user. In some embodiments, the array may be flexible so as to conform to the shape of the body portion of the user. The phased array may be shaped as a bi-lateral or two-lobed band 1641a, 1641b, such that the ultrasound beams may be directed around the frontal sinus. Alternatively, more than one array may be positioned on a body portion of a user, such that the arrays act in coordination as if they were a single array, as illustrated in FIG. 16B, showing a pair of ultrasound arrays 1661a, 1661b that are attached separately to different body regions but may be connected (e.g., by a flexible cable, etc.) and may share circuitry, including power supplies, controllers, etc. Removable markings, tattoos, or other identifiers may indicate the location at which the array should be positioned to receive the desired neuromodulation. The array may not turn on and/or function until the array is positioned in the correct location on the structure, device, or user. The array may identify a marker (e.g., a tattoo, an embedded chip, or another signal) on the skin of the user. Alternatively, the array may detect physical characteristics and/or features of the user's skin and bone structure, such that appropriate placement may be determined. A physician or another healthcare professional may need to position the array on the user before the array may be used, though the apparatuses may be configured so that additional (e.g. technician) assistance is not necessary.

FIG. 16C illustrates another example of an ultrasound phased array applicator device configured as a neurostimulator. In this example, the body of the device may be self-contained, and be relatively thin and lightweight. For example, the body may be thinner than 4 cm (e.g., thinner than 3 cm, and particularly thinner than 2 cm). The body may weigh less than a predetermined amount (e.g., less than 8 ounces, less than 7 ounces, less than 6 ounces, less than 5 ounces, less than 4 ounces, less than 3 ounces, less than 2 ounces, less than about 1.5 ounces, less than about 1 ounce, less than about 0.5 ounces, less than about 0.25 ounces). In FIG. 16C the body has a relatively round (disc-shaped) configuration, however in some variations the body may be alternatively shaped. In general, the skin-contacting surface may be curved or bendable, so that it may make contact (acoustic contact) with the wearer's skin. The contact surface may be referred to herein as a faceplate.

Any of the apparatuses described herein may include a moisture barrier, for example by using FR4 material and polyimide in a faceplate, such that sweating, water, and/or other moisture may not disrupt, short-circuit, or otherwise harm the phased array and/or the user. In some embodiments, a typical dosage level delivered by a wearable ultrasound phase array is between 0 watt/cm² and 2 watt/cm², preferably less than 1 watt/cm² time averaged. The number of cycles in a burst may be 1 to 500, preferably between 2 to 200. Pulse repetition frequency may be 0.5 Hz to 2 kHz, preferably 1 Hz to 1 kHz.

In some embodiments, a phased array may be used to measure, determine, specify, and/or otherwise collect data about the thickness of the skull bone of a user. Skull bone thickness data may be used as a security measure, such that each user has a unique skull bone thickness profile. Alternatively, skull bone thickness data may enable improved focusing, for example by determining aberrations from the nominal skull bone thickness and adjusting focusing of the array elements accordingly.

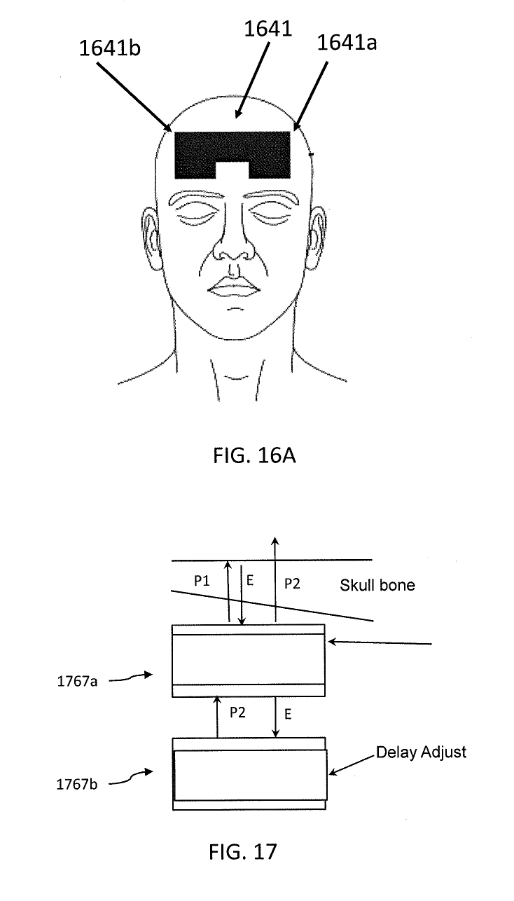

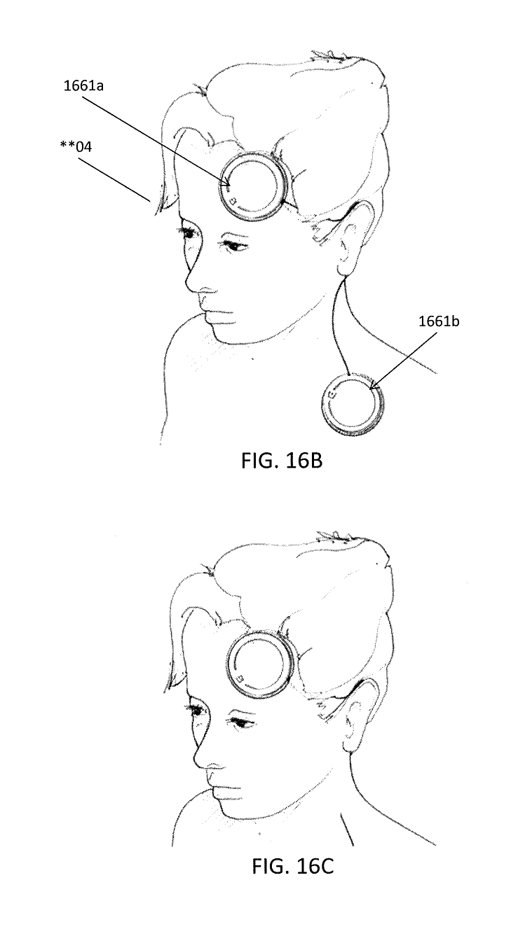

As mentioned above, in some variations, the apparatus may be configured to compensate for penetration through the tissue, and particularly bone (such as the skull) in delivering a predetermined dose of ultrasound energy. For example, FIG. 17 illustrates an example of an apparatus having least two array elements in the same module. The at least two transducer elements 1767a, 1767b may be positioned in the same module using SMT, as described above. As shown in FIG. 17, holding two or more transducer elements 1767a, 1767b in the same module may compensate for phase aberration effects. Phase aberration effects are due to spatial variations of tissue, structure, or device parameters that affect ultrasound propagation (aberration), and multiple reflections between tissue, structure, or device layers and the ultrasound array (reverberations). These artifacts can reduce the neuromodulation and/or imaging quality of the ultrasound phased array. As shown in FIG. 17, at least one transducer element 1767a sends out an ultrasound pulse P1 that generates an echo E from the tissue, structure, or device. In some embodiments, the echo E may trigger a second transducer element 1767b to transmit a pulse wave or pulse train P2 for neural modulation. The echo E from the tissue, structure, or device may adjust the transmit timing of the second transducer element 1767b, compensating for the phase effect of the tissue, structure, or device. For example, using at least two transducer elements 1767a, 1767b in the same module may be beneficial in reducing phase aberration effects from skull bone. The echo E from the thicker skull bone may adjust the transmit timing of the second transducer element 1767b, compensating for the phase effect of the thicker skull bone. The two (or more) transducers may be side-by-side relative to the energy delivery surface (e.g., adjacent to the skin), rather than overlapping.

FIG. 18 illustrates three angled array elements 1877 configured for ultrasound beam focusing. Single element transducers and traditional arrays are incapable of focusing at distances very close to the transducer face due to subtended angles from the focus to the transducer. In some instances, targeted neural pathways exist very close to the skull (e.g. cerebral cortex). In some embodiments, as shown in FIG. 18, angled array elements 1877 on the edge of the circuit board may be positioned at a fixed angle (e.g., by SMT), such that the array elements may reduce effects from subtended angles. In some embodiments, the angle θ of the array element 1877 may be between 0° and 180°, less than 180°, or equal to 180°. Alternatively or additionally, the transducer elements may be steered by using constructive/destructive interference (by beam forming), without necessarily holding them at a particular angle.

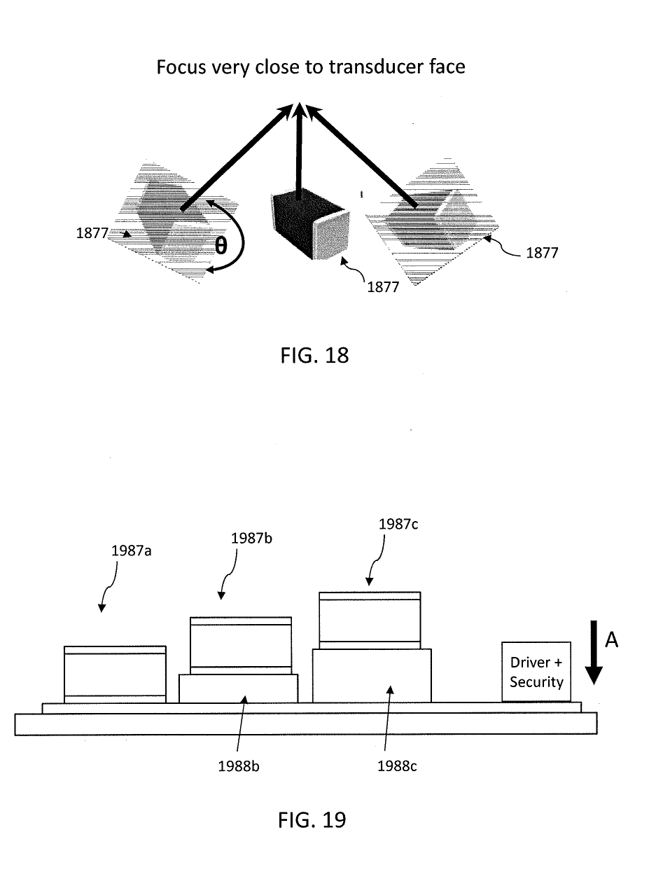

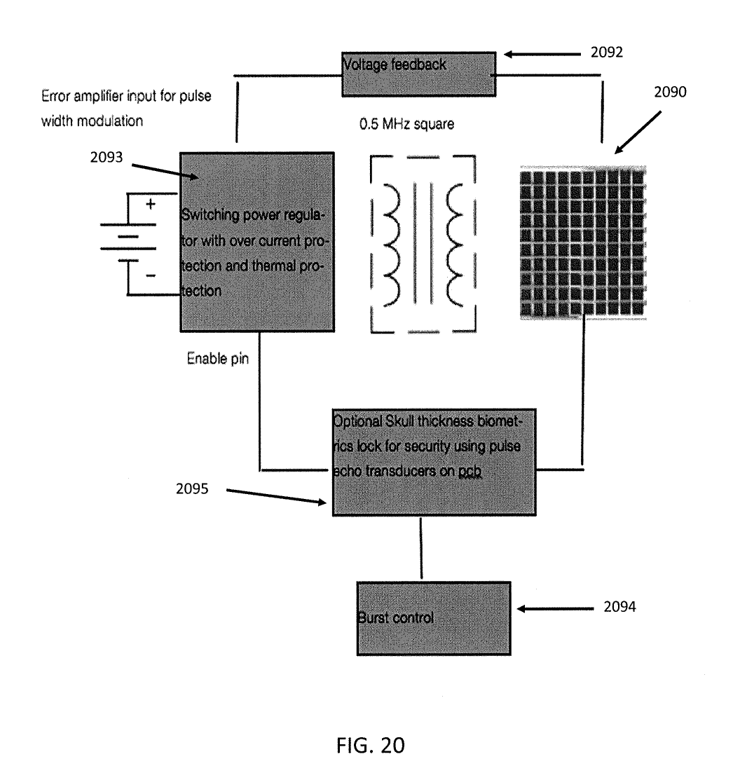

FIG. 19 illustrates an integrated time delay path and security model for a phased array. In some embodiments, array elements 1987b, 1987c may include an inherent time delay 1988b, 1988c, such that the ultrasound beam is steered and focused onto a specific target area. The array elements 1987a, 1987b, 1987c may be connected in parallel, such that only a single driver is required for all of the elements. As shown in FIG. 20, the driver 2093 may include a switching power regulator, configured to oscillate at the resonance frequency of the array 2090. The burst 2094 may be controlled by an enable pin of the driver 2093, such that only a specific number of cycles are transmitted at each session of the modulation. In some embodiments, an electrical transformer 2092, for example a flyback or line output transformer, connected to the driver 2093 may increase the voltage to control the power of ultrasound bursts so as to have a high peak power but low average power for neural modulation.

One or more thresholds, limits, and/or security parameters may be included to increase the safety of the phased array for commercial use. For example, a current limit may be set in the driver as a safety circuit to limit the power output of the array. Alternatively or additionally, a thermal circuit in the driver may be included as a second level of safety control. In some embodiments, the thermal circuit in the driver may alert the system and/or user when the temperature of the structure, device, and/or user increases more than 0.5° C. For example, the system may shut down, alter the excitation sequence, and/or deliver a signal (i.e. visual, audible, tactile, etc.) to alert the user to the temperature change. When an unsafe temperature is reached, the pulses may be paused until the thermal effect subsides.

For biometric security, a number of the ultrasound transducers may be operated in pulse echo mode to measure the skull thickness and express the thickness in a sequence of numbers, as described above. The sequence then represents the person's identity and may be linked or keyed to another biometric parameter of the user such as a fingerprint, ensuring that only the user can operate the array. Alternatively, another biometric of the user's anatomy (e.g. brain, head shape, etc.) may be used to identify the user. For example, electroencephalography pattern, skull diameter at several locations, and/or skull volume may be used as a biometric security parameter.

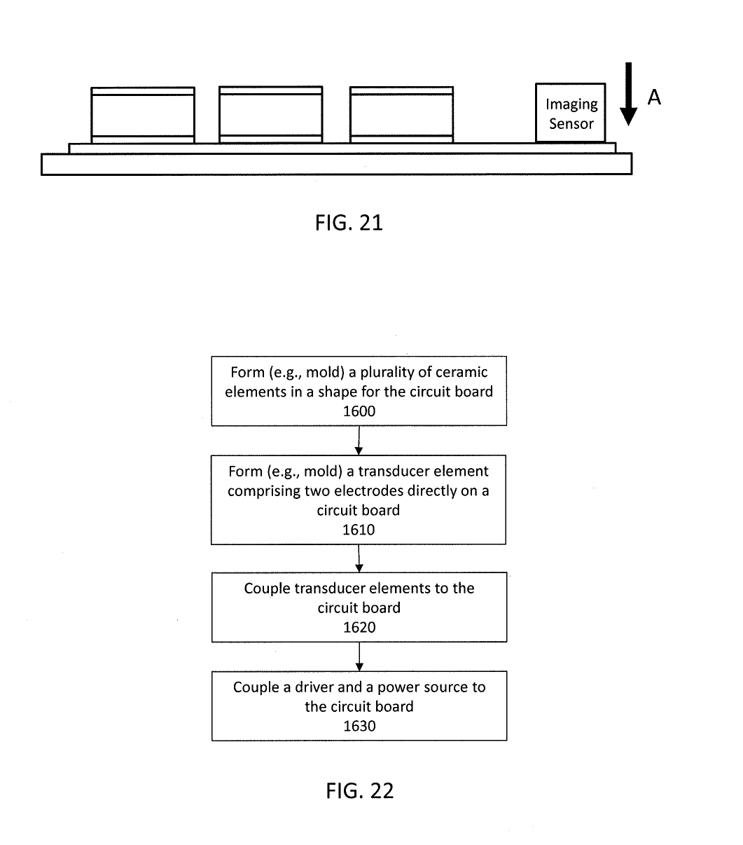

FIGS. 21 and 22 illustrate a system and method, respectively, for neuromodulation, using the apparatuses described herein. An ultrasound phased array may include an imaging sensor, as shown in FIG. 21. Alternatively, an external imaging device may be used before, after, and/or in parallel with use of an ultrasound phased array. In some embodiments, the imaging sensor may be used to monitor a condition, status, state, and/or natural functioning of a structure, device, or user. In some embodiments, the imaging sensor may be used to monitor and/or map a neural pathway, such that the ultrasound beam may map to the pathway, follow the pathway, or be timed to neural activity in a particular portion of the pathway. In some embodiments, the effect of ultrasound may be amplified several fold by multiple excitations along the same neural pathway in phase and in synchrony with the electrical signal.

The imaging sensor may form part of an array element transducer or phased array, as shown in FIG. 21. Alternatively, the imaging sensor may remain separate, such that the imaging sensor differs in frequency, efficiency, power handling, damping, and/or driver requirements. The imaging sensor may include Doppler, ultrasound tomography, non-linear cross beam, elastography, electroencephalography, fMRI, thermal impulse, and/or Lorentz movement. For example, using non-linear cross beam may reduce standing wave reverberations created by skull bone, which often blur traditional imaging. In non-linear cross beam ultrasound imaging, a new frequency or modulation different from the incident beam is generated where two ultrasound beams cross. The signal from the cross beam is made distinct from the incident beam due to the new frequency or modulation. Thus, the new signal being generated behind the skull may be less affected by the standing wave reverberations. Alternatively, ultrasound tomography imaging may be used to improve contrast, resolution and signal to noise ratio of imaging.

In general, a method of applying neurostimulation using a phased array ultrasound apparatus as described herein may include applying the apparatus to the skin, so that it may be worn. The thin apparatuses described herein are particularly beneficial, and may be applied to the subject's head, as illustrated above. In some thin and lightweight applications, the phased array is flexible in at least one dimension and can be adhered to a subject's head or body similar to a Band-Aid. Any of these methods may also include determining and/or defining the neural pathway to be modified. This may be done by imaging (e.g., by an imaging sensor), or by other means. Alternatively, the tissue may be scanning with the ultrasound beam, likely hitting the target region (other non-target regions may also receive ultrasound energy, but it may not prevent the effective use of the apparatus.). Alternatively or additionally, the ultrasound energy may then be steered and/or focused to the target neural pathway (e.g., in some variations defined by an imaging sensor). Neuromodulation of the neural pathway may then be evoked, e.g., by driving the array coupled to the user's head or other body portion to deliver ultrasound as indicated herein (e.g., between 20 kHz and 1 MHz, 20 kHz and 2 MHz, etc.). As mentioned, some variations of these methods may use an imaging sensor on a phased array or an external imaging means to determine, measure, and/or modify a neural pathway during delivery of ultrasound beams to a body portion of a user. However, these methods may be configured and/or adapted to be used in any field and to image any structure and/or device.

Any of these methods may determine and/or define a neural pathway to be modified using an imaging sensor. This step may preferably function to locate, map, discover, or otherwise find the neural pathway to be modified. Synchronizing the shape of the neural path, timing of the excitation, and spatial location of the sound beam may specify the neural pathway to be modulated. The shape of the neural pathway may define the neural function that the user desires to modify, enhance, or suppress.

Any of these methods may include steering and/or focusing the ultrasound beams to the neural pathway defined by an imaging sensor. For example, the steering and/or focusing step may function to steer and/or focus the ultrasound beams around the frontal sinus of the user and/or to specifically modulate a subset of neural pathways while maintaining surrounding neural pathways in their native state. Specifically, the array elements on the phased array may be positioned, angled, and/or otherwise modified to steer and/or focus the ultrasound waves, as described above.

These methods may include inducing neuromodulation of the neural pathway defined by the imaging sensor using the driver on the array coupled to the user's head or other body portion. Neuromodulation of the neural pathway may include delivering ultrasound waves to the neural pathway to enhance, suppress, and/or skew the effects of the neural pathway on the user. In some embodiments, multiple points along the same neural pathway may be simultaneously or sequentially targeted to synergistically affect the neural pathway. In some embodiments, the driver may be a square wave type or a switching regulator, as described above.

In some embodiments, the method may further include imaging and/or Doppler mapping of blood flow during neural stimulation using multiple frequency elements in the same array plane. Blood flow information may be used to determine the effect of neuromodulation on the user, a physiological and/or biological state of the user before, during, and/or after neuromodulation, and/or a condition of the user before, during, and/or after neuromodulation. The imaging and/or Doppler mapping step may further include using a very high frame rate of imaging, such that signal averaging can extract blood flow information from small voxels. Alternatively, the method may include determining the location of nerve bundles with the imaging sensor. In some instances, a nerve bundle location may have a spatial correlation with blood vessel locations.

In some embodiments, the method may further include saving and/or sending images to an external device. A user may desire to share, save, email, upload, and/or otherwise transmit information and/or images from a neuromodulation session to an external device, server, network, social media site, physician, friend, or acquaintance. The external device may be a mobile device, laptop, desktop computer, server, or any other suitable device. The information and/or images may be sent via a cable, Bluetooth, Wi-Fi, or any other means to an external device. Alternatively, the array may include internal storage, such as a flash drive, Secure Digital card, and/or optical disc. In some embodiments, the array may include a USB port, an IEEE 1394 interface, or any other type of port for transmitting, sharing, saving, or otherwise using data collected by the phased array. The device may include software and/or hardware for viewing, reconstructing, editing, and/or otherwise modifying and/or viewing the information and/or images.

Any of these methods may further include delivering a therapy before, during, and/or after neuromodulation. In some embodiments, a therapy element may be coupled to the array, as described above. Alternatively, in the some embodiments, the therapy may be delivered independently of the phased array, for example by an external means. The external means may include delivery orally, intravenously, intraperitoneally, intrarectally, intradermally, or any other recognized delivery method. In some embodiments, the therapy may include an injection, pill, food, drink, gas, biologic agent, recombinant agent, naturally occurring agent, or any combination thereof.

These methods may further include determining an identity of a user by measuring skull thickness, measuring skull volume, acquiring fingerprints of the user, and/or measuring any other biometric parameter of the user, as described above. In some embodiments, the phased array may be a prescription device, such that the phased array may only be turned on and/or used after the correct user is identified. In some embodiments, images and/or information may be shared, emailed, uploaded, saved, and/or otherwise transmitted only after the user is identified, as described above.

As mentioned above, any of the apparatuses described herein may be included as part of a system or device. For example, any of these apparatuses may include one or more wearable devices including a phased array of ultrasound transducers. These devices may be referred to as ultrasound phased array applicators. A system including an ultrasound phased array applicator may be configured to wirelessly communicate (and/or in some variations include) a user computing device that may be used to control and/or modify the activity of the ultrasound phased array applicator device. In some variations the user computing device (which may be, e.g., a phone such as a smartphone, a desktop, a tablet, a laptop, etc.) is configured to provide stimulation parameters to the ultrasound phased array applicator device, such as applied energy (e.g., waveforms to be applied, steering information, power level of stimulation, timing/dosing regimes, etc.).

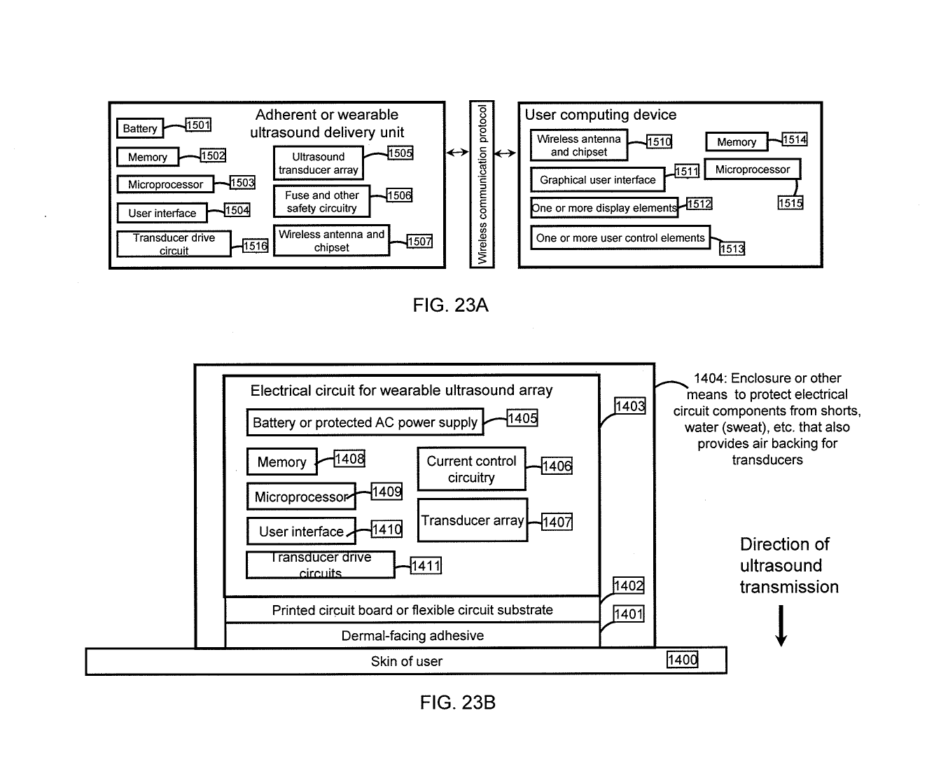

For example, FIG. 23A illustrates one example of a system including an ultrasound phased array applicator device that wirelessly communicates with a user computing device running control logic (e.g., including control circuitry, hardware, firmware, and/or software) to control and/or record or guide application of ultrasound stimulation from the ultrasound phased array applicator device. In FIG. 23A, the ultrasound phased array applicator device (referred to as an adherent or wearable ultrasound delivery unit) includes a battery 1501 (or any other power supply, as described above, including a port, plug, or cord for connecting to an external (i.e. AC) power source), a memory 1502, a processor 1503, a user interface (which may include a display or output, including LEDs, a display screen, etc.), a fuse and other safety circuitry 1506, as well as a wireless antenna and chipset 1507. Any of these variations typically includes an array of ultrasound transducer elements 1505, such as those described above. The devices may also include a transducer drive circuit for applying power to the transducer elements to deliver ultrasound. The controller and/or the drive circuitry may also be configured to steer the beam (beam form). The drive circuitry may include any appropriate circuit element, including amplifiers, resistors, or the like. The user computing device may control the function of the drive circuitry and other components of the phased array (e.g., the components may be controlled by software or other non-transient signals that cause the computing device to communicate with the ultrasound phased array applicator device). For example, the controlling logic may be configured to control the operation of the wireless antenna chipset 1510 from the user computing device, provide a graphical user interface (GUI) 1511, and one or more display elements 1513, and/or one or more control elements 1513, a memory 1514, and processor 1515.

FIG. 23B shows another variation of an ultrasound phased array applicator that may be used. For example, in FIG. 23B, the ultrasound phased array applicator device includes the transducer array 1407, and drive circuitry (e.g., transducer drive circuits 1411, control circuitry 1406, a memory 1408, power source 1405, processor 1409, display/user interface 1410, etc.), this material may be attached to a printed circuit board (e.g., rigid PCB or flexible substrate 1402), as described above. The device may include a patient-contacting surface, which may be the back side of the PCB 1402, and may include an adhesive 1401 for acoustic (and in some variations, mechanical) coupling to the patient's skin 1400. The entire apparatus may include a covering o housing to protect the apparatus 1404.

### Examples

Described below are examples of the apparatuses and methods described above. Any of these examples may include one or more features or elements that may be incorporated or included in any other apparatus and method described herein, unless specifically indicated otherwise.

Neural stimulation and neural modification using ultrasound energy, which may include the suppression or enhancement of neural activities already in existence and/or the generation or triggering of new neural activities not in existence before the stimulating event, may be performed using any of the apparatuses described herein. For example, inducing a change in enhancement of neural activity and capability may be performed by ultrasound to enhance sensory capability. The apparatuses and methods described herein for ultrasound neuromodulation may induce a relaxing, calming, anxiolytic, dissociated, high mental clarity, or worry-free state of mind in a subject that would be advantageous for improving the subject's experiences and state of mind, as well as addressing insomnia and mitigating negative responses to stress. Similarly the apparatuses and methods described herein for ultrasound neuromodulation may increase a subject's motivation, subjective (and/or physiological) energy level, or focus and would be advantageous for improving a subject's productivity and providing beneficial states of mind.

Ultrasound delivered by any of these apparatuses may be focused to a fairly small spot size, even quite close to the transducer (near field) using the apparatuses described herein. The use of ultrasound has the benefit of being selective in the effect on neural pathways by spatial location. In essence, ultrasound can perform and achieve in some cases as if electrodes were implanted in the brain, except without the need for surgery. The fairly low ultrasound level required to achieve neural modification using the apparatuses described herein opens the possibility for use of ultrasound in the consumer space, particularly for non-invasive and functionally selective ultrasound apparatuses to achieve cognitive effects by neural modification, stimulation, or inhibition effects. Since ultrasound travels faster than neural signal propagation, ultrasound's modification effects can be triggered by a neural stimulation event and modulate the induced effect on the brain (and, more generally, nervous system) by stimulating areas before or as the endogenous neural pathways are activated by the triggered event, for example, a signal may be time locked to a hearing aid, or to music, or to a lecture, or to a glucose monitor, or to a communication device such as a cellphone, or to a learning device, such as an electronic book. The modulation effect of ultrasound provides an emphasis/de-emphasis/focus of the information from the external device based on affecting neural circuit function at one or more sites activated, inhibited, or modulated by an event related to the information of the external device (which may, in some embodiments, be information acquired by a sensor component of the ultrasound array apparatus). Further, the general enhancement of neural activities may have an empirically demonstrable net effect on motivation. This subtle and noticeable benefit works very well with wearable devices which provide feedback to the user of their physiological states, such as blood glucose level. Typically a user who noticed an increase in blood sugar knows that action should be taken, such as starting to exercise to burn the extra sugar in the blood. However compliance has been low in the motivation to take action. By stimulating relevant brain areas with targeted ultrasound neuromodulation using the apparatuses and methods described herein, direct cognitive effects may be triggered that circumvent intrinsic limitations to guide adaptive behaviors.

As another area of benefit, based on the ability of ultrasound neuromodulation to suppress neural propagation, the apparatuses described herein may aid in pain control from the central nervous system and/or in the peripheral nervous system. Further, although the devices described herein are primarily intended as non-invasive apparatuses, we do not exclude the invasive use of ultrasound for the purpose of neural modification. For example, the use of ultrasound in an implant for the purpose of continued or as-needed neural modification may be performed using any of the apparatuses described herein, and in particular the ultrasound phased array applicators.

We described herein that in addition to the ability to focus, ultrasound can also be steered using a phased array system to follow the propagation of an impulse. This technology is possible because ultrasound propagates at a higher speed than neural propagation. We can therefore track propagation of a nerve impulse in real time and stimulate at multiple spatial points along the pathway of propagation to directly modulate the effect of that nerve impulse on downstream pathways. If the timing of stimulation is controlled precisely, the effect of tracking will be similar to a “parametric amplifier” where a gain may be achieved by pumping at several points along the signal pathway in sync and in phase with the signal's propagation. Ultrasound may therefore be made more precise in the selection of neural function in combination with a neural recording function and a strategy of ultrasound delivery with a pattern of steering that follows the intrinsic nerve impulse propagation, so as to interact at more than one point with the neural event in synchrony with the impulse propagation. This may allow tracking of neural signal propagation, either for determining the neural pathway in a person (personalized brain mapping), or in creating a multiplying effect of ultrasound's modulation effect.

An object of the apparatuses and method described herein is to make these arrays inexpensively. A third objective is to provide a form factor conducive to putting the array as a head-band or as a Band-Aid shaped adhesive patch. Thus, described above, and in examples below are: methods of fabrication of array by SMT; the use of lateral mode resonance to make very thin arrays; methods to steer and focus with time delay built into array elements; methods to drive the array at low cost; and methods to track and stimulate along neural propagation path to specify a function for modification.

The ultrasound phased arrays described herein (particularly as versus a single ultrasound transducer) include the ability to easily steer and focus energy spatially, in a way similar to a phased array radar. As a result of having a phased array, the neural modification can be made functionally specific, allowing lower dose level, higher gains in effect, and/or stimulation of deep structures without substantially affecting more brain regions more proximal to the phased array. This specificity may be achieved by spatially pointing the sound beam to a specific location of the brain that affects the function.

By the use of a phased array, and especially one that is two dimensional (2D), these apparatuses may allow the ability to steer both in the X and Y direction as well as focusing at various focal depths, even though the ultrasound array is fixed for various targets. The ultrasound beam emitted (which may include side lobes) may be wider than the neural target.

In the case of a prescription device, the ability of the array to steer and focus may allow a health care professional to mark on the user's skin the location for placement of an adhesive thin ultrasound phased array. As long as the shaped array is placed correctly by the skilled practitioner of ultrasound neuromodulation (macro-positioning), the sound can be steered and focused to the desired location to achieve the goal of the prescription (micro-positioning byb beam-steering and focusing). The array apparatuses described here may also achieve a de-focusing effect as needed in some circumstances, as in the case of wanting a general enhancement or suppression of neural activity, where the steering and focusing of sound can be made deliberately and controllably broad. For example, increasing the excitability of a portion of cerebral cortex may modulate the effect of endogenous activity, such as stimulating all of primary somatosensory cortex to increase the sensitivity of the perception of touch. Prescription devices for the stimulation of single or multiple brain areas can be generated either in a software or hardware system. Examples of software prescriptions would be inclusion of phasing information that is stored on a controlling device that generates or reroutes appropriate phases dynamically to individual elements. These prescriptions can be dynamic in their stimulation (e.g. stimulate two brain regions sequentially). Hardware prescriptions may come in a form similar to an SD card or a small PCB circuit board that can statically route appropriate phases to specific transducer elements, or may be a software program stored on a durable, machine-readable portion of the device, or transmitted wirelessly (e.g. by Bluetooth or Wi-fi) to the ultrasound phased array controller. In other embodiments, fixed focusing may be performed by 3D printing of adapters/couplants that include appropriate delays specific to an individual's anatomy

Unlike the ultrasound elements described herein, traditional arrays are made by dicing after a piezoelectric material is coupled to other components (i.e. back material). Although dicing is one means for creating appropriately sized and shaped transducer elements of the current invention, the dicing will be a first step before the piezo material is connected to other material. For example, a traditional ultrasound array is typically made by potting a large piezo plate into a backing material or a matching layer, then dicing the ceramic plate into smaller elements. Once the elements are defined, connections are made to the elements manually or semi-automatically. Matching layers, lens, backing, front plates, EMI shielding, electrical connections, moisture barrier, and other mechanical structures are bonded consecutively onto the array to make the finished product. Such traditional arrays have low yield and are expensive, thick, heavy, and/or rigid. Because of mechanical impact in the dicing step, the yield of traditional arrays is hard to control. A mechanically damaged element can ruin an entire array. Due to the poor yield, traditional arrays are very expensive. A one-dimensional array may cost upwards of several thousand dollars. A 2D array may cost even more. Thus, the resulting traditional device is typically rigid, fairly thick, weighs a lot, and expensive. Further, traditional arrays are not conducive to customization for individual users. A traditional array also takes several days to finish production because of the many steps to attach, glue, epoxy bind, and encapsulate the various components by hand to the array. Special fixtures and jigs are required for each of the steps for every model of an array.

In contrast the ultrasound phased array applicators described herein may not have any of these disadvantages. The methods of array construction described herein may use a method of construction that results in a light weight, thin, flexible (as needed), and low cost apparatus. These characteristics allow the array to go on the user as an adhesive bandage (e.g. Band-Aid), or as a headband, or in a form that enables wearability. For example, the number of elements in an array is proportional to its size or aperture. For neural modification, the array may need to cover a large area so that the neural target of interest is within coverage of the array. An array for neurostimulation may of deep brain targets may need a large aperture. The large size may mean a lot of elements are required for neural modification. The yield of the array must not suffer despite the large number of elements. The traditional array fabrication does not meet these demands.

Described herein are methods including Surface Mount Technology (SMT) used regularly for manufacture of electronics, particularly compact, light-weight, and miniature electronics, including mobile phones. Many (up to hundreds to thousands) of transducer elements in a phased array may be treated the same and placed precisely on a substrate (e.g., PCB). FIG. 2, above, shows array elements loaded onto an SMT tray ready for pick and place mounting onto a printed circuit board or other (e.g. flexible) electronic circuit substrate. In SMT, miniature components with flat surfaces are pick-and-placed by a precision robot onto a printed circuit board (PCB), which can be flexible or rigid. On the PCB, right before pick and place, a solder paste may be deposited onto the PCB with a template called the stencil, a thin metallic sheet laser machined with precision openings. The thickness of the stencil determines the thickness of the solder paste. To achieve the best ultrasound performance, the stencil may be made very thin for the array construction. Because piezo ceramics typically have a low Curie temperature of around 220° C., regular solder paste cannot be used to manufacture phased arrays. A special formulation that melts at approximately 160° C. may be used for the array SMT. Once the components are placed on the PCB, the PCB may be heated (e.g., by going through a tube oven), to melt the solder paste into an alloy film that bonds with the component mechanically and electrically. In designs where ultrasound goes through the solder joint and the PCB before exiting the array, a vapor phase reflow may be used to extract any bubbles that may form in the solder paste during the reflow process. In the vapor phase reflow process, a vacuum is applied during the reflow. As mentioned above, an automatic intelligent vision system may then examine the loaded PCB for variances in orientation or position to a very high accuracy. A worker will rework the PCB assembly if there are defects found, or reject the assembly if rework is not possible.

The assembly that comes through this process typically has a high quality because of the complete automation, intelligent optical inspection, and the precise process control built into the process. SMT often achieves quality levels of 10 defect parts in a million or better. The finished PCB may then be put into an automated tester where miniature probes contact the PCB to verify function and reliability of the PCB assembly. Selected samples may be placed into more sophisticate testing to assure compliance to reliability and quality goals. Fast production cycle time may also enable prescription-based arrays (e.g., custom arrays for individual users or classes of users). Further, the SMT approach also allows a very fast turnaround of a new array design. A new design may be achieved by a new layout of the PCB and a reprogramming of the robot, which can be done a lot faster than fabricating a new series of manufacturing tools and fixtures as required in a traditional array.

The ultrasound elements described herein may use any appropriate Piezo ceramic such as lead zirconium titanate, for example PZT4 or PZT8, Lead Metaniobate, or composites such as PZT4 pillars encapsulated in an epoxy matrix. The choice of piezo material may depend on the acoustic impedance and drive voltage desired for driving the transducer.

The apparatuses and methods described herein may also be configured to operate primarily (e.g., greater than 50%) in a particular resonance modes. For very thin arrays, the apparatus may use a radial mode resonance mode, where width to thickness may be greater than 2 (e.g., greater than 3, 4, 5, 6, 7, 8, 9, 10, or between about 1, 2, 3, 4, or 5 and 20, 15, or 10, etc., including between about 1 to 20). The use of radial mode resonance in a phased array as described herein is unusual. Traditional array elements have a width to thickness ratio (the aspect ratio) between 0.4 to 0.6, and avoid radial mode transmission because of the losses in efficiency. However, the traditional thicker array elements are unsuitable for thin phased arrays described herein. Surprisingly, the need for a very thin profile can be met by radial mode resonances without causing problems due to the low efficiency, particularly when stimulating (e.g., and not imaging). Radial mode transducers may set the resonance frequency by the width and length of the array element, rather than the thickness. Therefore the elements can be made very thin.

For arrays requiring high efficiency, such as very small sized battery powered applications, cubic array elements may be used, or transducer elements approaching cubic dimensions. Surprisingly, an array element's efficiency jumps up substantially, and is the best, when all 3 sides of the elements are the same. This is because the resonance frequency in each of the dimensions is equal. Since in a wearable application, the array may be powered from a battery, efficiency may be important.

The electrode pattern of the array element may align the element on the PCB. Once the solder paste has melted, the element may “float” on top of the molten alloy. The surface tension of the electrode against the pad may then align the part until the PCB cools off and the solder solidifies. The traditional array elements have electrodes on the top and bottom surfaces of the element, where the top is typically a common ground and it faces the user. This electrode pattern will work for the apparatuses described herein, however such transducers may have a tendency to rotate or shift during fabrication. This may be corrected by using a solder resist film that is slightly taller than the solder thickness around the PCB pad (in order to provide a return ground, a film with a metallic coating may be required to cover multiple elements, and a conductive compound such as silver epoxy creates an electrical contact between the array element's electrode and the film which serves as common ground).

In some variations, a second electrode, typically a ground, may wrap around the side wall of the array element to go to the same plane as the first electrode so that both electrodes make contact with the PCB pad. This pattern may improve the drifting of the element during reflow and eliminates the need for a common ground metal film. The end cap electrode (the first electrode) may be included and may help self-align the transducer during reflow. Both electrodes of the ultrasound element may run parallel to the direction of the sound propagation towards the user. In some configurations the ultrasound does not pass through the electrodes; for example, lateral compression of the piezo ceramic due to electric field from the end cap electrodes may cause the thickness of the ceramic to change due to the Poisson ratio of the ceramic.

The current manufacturing method also allows the use of elements optimized for different ultrasound frequencies, allowing the use of further spatial shaping using beat patterns and other frequency mixing techniques. Multiple frequencies may be simultaneously used with high electrical efficiently if different array elements tuned to different center frequencies are on the same system. For example, the shaping of the flexible PCB boards can be used in addition to create natural focus or foci instead of or in addition to phasing capabilities. Control of the aperture may also be done electronically, increasing or decreasing the aperture as needed. In any of the variations described herein, one or more sensors for detecting the flexing of the PCB may be used to determine the shape and current location of individual array elements as the PCB is bent to fit the application surface such as on a user's forehead or arm. For example, optical sensing may be used. Strobing or different colored LEDs (particularly in the IR region) can be used in addition to triangulate the three dimensional spatial locations of the array elements. The control circuitry and the LEDs themselves could be trivially integrated using the outlined manufacturing method. A simple webcam or phone camera can be used to track these, along with standard facial anatomical landmarks to define a position relative to facial anatomy.

As mentioned above, the transducer array elements may be positioned using suitable anatomical landmarks (using facial recognition and analysis through camera images, or by placing spatial sensors or LEDs at reference positions such as the tip of the nose or base of the ear), as well as location and tilt information (as measured by accelerometers, gyroscopes, etc.), in conjunction with facial image analysis performed using a user computing device with a simple smartphone camera or webcam. These individualized positions could be used in conjunction with averaged anatomical brain maps that are stretched and transformed to the individual, or with DICOM images from MRI, PET, CT, or other medical imaging modalities. In addition, ultrasound imaging sensors of the apparatus may be used to detect skull thickness or image brain morphology and vasculature for alignment with DICOM and standard anatomical images.

Intermixed ultrasound receivers can be used to determine both the spatial locations as well as aberrations caused by differences in the thickness of the skull by activation of individual or small patches of ultrasound array elements and measuring the time until these “pings” are detected by the various receivers to create a mapping for the ultrasound elements.

In any of the methods described herein, in addition to using bulk single materials and static mixtures as couplants of ultrasound, other materials with more dynamic properties may be used. The simplest is the use of evaporating, low-residue liquid as a couplant such as an alcohol based solution. Similarly, biological compatible liquids that may be absorbed by the scalp may be used. Magnetic fluids could be used as well to create an interface between the array probe and scalp. These solutions allow low residue or clean removal of couplants that can help efficient ultrasound transmission even through hair. Alternately, static but micro-structured couplings may be added to the transducer array elements such that they can go through and transmit ultrasonic energy to the scalp, bypassing the hair, much like a brush or comb's bristles. These couplings could be made of a number of materials with suitable acoustic impedance properties intermediate to that of the piezo element and scalp, including but not limited to silicone, polyurethane, polyethylense, graphite, aluminum, carbon fiber, and carbon nanotubes. These couplings also need not all be of the same length, allowing the possibility of introducing delay lines for different array elements as well as for individual array elements. In addition, more advanced materials that change their shape, molecular or crystalline structure, and/or acoustic impedance when temperature, pressure, electrical, magnetic, or electromagnetic fields are applied can be utilized for dynamic control. Non-Newtonian fluids, ferrofluids, magnetorheological fluids, electrorheological fluids, and electromagnetorheological fluids are examples of such materials.

In general, driving circuitry for a phased element can be broken down into two basic methods. First, each element could have completely independent and programmable timing. Second, the timing of each element could be matched to one of a plurality (e.g., 2 to 100, 2 to 75, 2 to 50, 2 to 40, 2 to 30, 2 to 25, etc.) phase signals at the ultrasound frequency (e.g., for 1 MHz ultrasound, a 2 phase signal means one signal that is at 1 MHz, and another 1 MHz signal that is exactly 500 ns shifted in time.) Previous studies have shown that at frequencies applicable for transcranial ultrasound neuromodulation, simply using 4 different phase options for the elements can recreate focal points with up to 80% of the power one would obtain at the focus if each transducer had fully optimized delay times and phases. Three phases could still achieve close to 65% of the power, with 2 phases allowing 40% of the possible peak power. Timing signals for both methods can be generated digitally by use of reference clocks at the ultrasound frequency, at multiples of the ultrasound frequency, or at a sufficiently larger frequency such that a rational fraction allows close approximation of target ultrasound frequency, and counters and/or delay lines. The first method will be described in the first paragraph following, while the multiple strategies for the second method will be described in paragraphs that follow. Each array element may be part of a module that may perform one or more of the following functions: generate ultrasound frequencies, derive ultrasound frequencies and delays from a higher speed clock, create delayed timing signals from a reference clock, convert low-voltage and low-current signals into higher voltages to drive transducer elements, or convert low-voltage and high-current signals into higher voltages to drive transducer elements. The components of the module are uniquely associated to one or patches of transducer array elements, but need not be in physical proximity to the elements themselves as long as they are independently electrically connected.

Timing can be generated by a number of methods. In one embodiment, each module could have a programmable clock that can generate low voltage (e.g., <20V) pulses at the ultrasound frequencies and with a reliable delay time relative to a start trigger. All modules may receive a universal timing trigger so that they are in the correct phase or delay relative to each other. In one embodiment, each module could receive the same master clock signal at the ultrasound frequency, and will create a delayed timing signal using a delay line or similar means. In another embodiment, each module could receive a reference clock that is some multiple of the ultrasound frequency, and programmable counters will be used to create delayed timing signals at the ultrasound frequencies, with resolution dependent on the counter and the reference clock. To amplify the low voltage signals, each of these timing signals would be connected, potentially through a combination of logic level shifters and FET drivers, to a single N-channel MOSFET, a single P-channel MOSFET, an N&P pair of MOSFETs, half or full-H bridge, class D and E RF amplifier circuitry, integrated ultrasound pulser chips, or voltage-controlled oscillator or phase-locked loop circuitry constructed with components to directly generate frequencies at high voltages, in order to generate a higher voltage necessary to drive the array element. The simplest embodiment would utilize a single positive and/or negative voltage rail. However, each array could also select from a number of transducer element driving voltages if there are multiple voltage rails available. Some of these elements need not be physically close to the array element itself, such as the timing generator, as long as independent connections to each array element exist.

Each module could connect to one of the plurality of low-voltage, low-current capacity phase signals using a digitally controlled switch. The voltage can then be amplified using the same techniques as described in the previous method, e.g., to amplify the low voltage signals, each of these timing/phasing signals may be connected, potentially through a combination of logic level shifters and FET drivers, to a single N-channel MOSFET, a single P-channel MOSFET, an N&P pair of MOSFETs, half or full-H bridge, class D and E RF amplifier circuitry, or voltage-controlled oscillator or phase-locked loop circuitry constructed with components to directly generate frequencies at high voltages, in order to generate a higher voltage necessary to drive the array element. The simplest embodiment would utilize a single positive and/or negative voltage rail. However, each array could also select from a number of transducer element driving voltages if there are multiple voltage rails available.

Each module could connect to one of the plurality of low-voltage, high-current capacity phase signals using a digitally controlled switch. These low-voltage, high-current phase signals could be used to directly drive high-voltage MOSFETs with large gate charges which drive the transducer elements, or be transformed into high-voltage, low-current ultrasound driving waveforms using a toroid transformer.

Each element could directly connect to one of the plurality of high-voltage, moderate-current capacity phase signal sources capable of directly driving ultrasound array elements.

In general, the methods and apparatuses described herein may be used with any other stimulation (e.g., neurostimulation) techniques. For example, electrical stimulation modalities (tDCS, tACS, tES, pulsed transdermal electrical stimulation) have been shown to be effective brain modulation modalities, particularly for cortical neurons and peripheral nerves. Magnetic stimulation (TMS) is also a powerful modality for superficial neural targets. Electromagnetic radiation (infrared light, terahertz waves, etc.) has also been shown to modify neural activity. The ultrasound methods and apparatuses described herein allow for the creation of sparse arrays into which electrical stimulation electrodes and EM generators (e.g. LEDs and microantennas) can be integrated onto the same control board (PCB, flexible circuit, etc.). In addition, couplants for ultrasound can be made electrically conductive and transparent to the EM wavelengths used in order to simplify the manufacturing process and reduce costs. These stimulation modalities may be interspersed with ultrasound, or used in conjunction (e.g. simultaneous magnetic field with ultrasound to shape stimulation sites or to lower the necessary ultrasound power for an effect.)

### Example

### Phased Array of Radial Mode Ultrasound

FIGS. 24-27 illustrate one example of an apparatus as described above, built and tested. In this example, the apparatus was formed by a pick and place method, with reflow, as described above, for 100 transducer elements. The array elements (transducer elements) were formed and held in a tray for placement by a robot using a suction method. As shown in FIG. 24, an exemplary PCB was fabricated with traces indicating locations (squares) for placement of the transducer elements; transducer elements were held to the pads and connected to traces on the active and ground electrodes for each transducer. A low temperature solder paste that melts at 140° C. was used along with a low heat (max oven temperature at 160° C.).

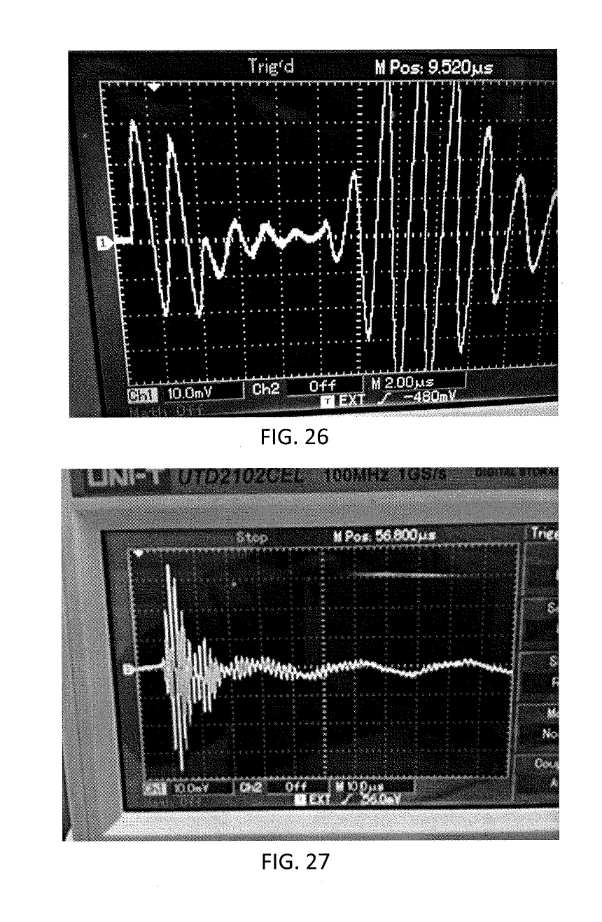

The array of transducer elements was coupled directly to circuitry on the PCB, including driver and power circuitry. In this working prototype, a Buck switching regulator (illustrated in FIG. 25) that puts out about 180 volts p-p, was used to drive signals across the transducer elements. In this example, a small tuning coil was used to keep the surge current in check. In testing, the power supply (battery) registered 11 Watts of electrical power consumption at 180 volts. The prototype switching circuitry drove the array, as illustrated in FIG. 26, showing a waveform from the printed circuit board side, i.e. through the FR4 (PCB) acting as a matching layer. The amplitude through the matching layer was at least twice as high in intensity as the amplitude through the opposite side of the apparatus, greater matching (tuning) of the PCB may allow greater gains. FIG. 27 shows an example of the acoustic signal delivered by the apparatus (shown as the Gaussian enveloped signal starting at the 3rd division from the right). In this example, an air-coupled transducer may efficiently transmit the ultrasound through the skin to the user. Since air has a very low density, the impedance mismatch between air and skin will cause some of the ultrasound energy to reflect off the skin. To optimize the energy transmission, a matching layer with acoustic impedance between air and skin can be placed on the user. For example as a passive material (adhesive, etc.), or part of a clothing accessory such as a head band of an appropriate thickness and material may be used to which the apparatus could be applied. The matching layer may be of quarter wavelength thickness, though it is not necessary the case. For a 0.5 MHz ultrasound frequency, the matching layer applied to the skin may be, for example, one mm or thinner. An example of the material to be used may be glass micro-balloons in a silicone matrix, but other material with a low density can be used. In some variations a dry coupling medium of a density higher than air but lower than water may be a good compromise between ergonomics and energy efficiency. The dry coupling medium may be applied to the user as an adhesive, gel, or as part of a clothing accessory. The ultrasound transducer apparatus may be optimized to couple to the acoustic impedance of this dry matching medium. For example, a dry coupling medium may contain glass micro-balloons and a silicone filler, or be made of other solids with a low density, such as aerogel which is a synthetic porous material in which the liquid component of the gel is replaced by air.

In some variations, it may be desirable to couple ultrasound to a user for neural modulation without the need of gel or creams. User acceptance may be higher when there is less complexity in the set up. However, particularly in larger-surface area apparatuses, it may be desirable to allow the apparatus to conform tightly to the skin, to avoid regions where trapped air (bubbles) may prevent good acoustic coupling with the skin, creating a high attenuation path affecting the coupling of ultrasound. The apparatuses described herein use a phased array, which does not require a large size mechanical lens for focus. Individual elements of these phased arrays are typically 2 mm in size or smaller. In some variations, each array element may be coated with a hemispherical polymer coupling cap and mounting the array elements on a flexible circuit board so that the array conforms to the user's anatomy, to achieve good coupling without the use of gel or cream. The stiffness of the flexible circuit board may thus be chosen so as to provides an appropriate level of contact force. Thus, an array elements and flexible PCB may be used and configured to bottom out on a preformed mandrel of known curvature so as to allow accurate control of focusing and steering. For example, with a hemispherical polymer coupling cap, very low force is required on the array element to achieve good coupling; the hemispherical shape pushes air away from the contact surfaces. Thus, a polymer hemispherical cap on the array element may permit the apparatus to be used with very little or no force required to achieve adequate dry coupling. The shape, height, and/or mechanical compliance of the coupling cap maybe improved to achieve even better performance.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1.-30. (canceled)

31. A wearable phased array ultrasound neurostimulator apparatus, the apparatus comprising:
an array of ultrasound transducer elements wherein each transducer element has a width and a thickness, and the width is greater than twice the thickness to cause preferential operation of said transducer element in a radial mode as compared with a thickness mode;
control circuitry configured to operate the array of ultrasound transducer elements between 100 kHz and 1 MHz as a phased array to cause a beamforming of emitted ultrasound signals; and
an outer surface of the apparatus configured to adhesively attach to a subject's skin.

32. The apparatus of claim 31, wherein the spacing between the ultrasound transducer elements of the array of ultrasound elements is between about 0.2 mm and 2 mm.

33. The apparatus of claim 31, wherein the control circuitry is printed onto a printed circuit board (PCB) substrate configured as a matching layer, a front side of each ultrasound transducer element of the array of ultrasound transducer elements being mounted onto said PCB substrate to cause a transmission of the emitted ultrasound signals through the PCB substrate.

34. The apparatus of claim 31, wherein the array of ultrasound transducers are air backed on a back side of each ultrasound transducer element.

35. The apparatus of claim 31, wherein the width is between about 2 times and 10 times the thickness for each transducer element.

36. The apparatus of claim 31, wherein the width is approximately half of a wavelength of an ultrasound signal emitted by the apparatus.

37. The apparatus of claim 31, wherein the thickness is 25% or less of a wavelength of an ultrasound signal emitted by the apparatus.

38. A wearable phased array ultrasound apparatus, the apparatus comprising:
an array of ultrasound transducer elements wherein each transducer element has a width and a thickness, and the width is greater than twice the thickness to cause preferential operation of said transducer element in a radial mode as compared with a thickness mode;
control circuitry configured to operate the array of ultrasound transducer elements between 100 kHz and 1 MHz as a phased array to cause a beamforming of emitted ultrasound signals; and
an outer surface of the apparatus configured to be acoustically coupled to a subject's skin.

39. The apparatus of claim 38, wherein a spacing between the ultrasound transducer elements of the array of ultrasound elements is between about 0.2 mm and 2 mm.

40. The apparatus of claim 38, wherein the control circuitry is printed onto a printed circuit board (PCB) substrate configured as a matching layer, a front side of each ultrasound transducer element of the array of ultrasound transducer elements being mounted onto said PCB substrate to cause a transmission of the emitted ultrasound signals through the PCB substrate.

41. The apparatus of claim 38, wherein the array of ultrasound transducers are air backed on a back side of each ultrasound transducer element.

42. The apparatus of claim 38, wherein the width is between about 2 times and 10 times the thickness for each transducer element.

43. The apparatus of claim 38, wherein the width is approximately half of a wavelength of an ultrasound signal emitted by the apparatus.

44. The apparatus of claim 38, wherein the thickness is 25% or less of a wavelength of an ultrasound signal emitted by the apparatus.

45. A wearable phased array ultrasound neurostimulator apparatus, the apparatus comprising:
an array of ultrasound transducer elements wherein each ultrasound transducer element has a width of between about 0.5 mm and 2 mm and a thickness, and wherein the width is greater than twice the thickness, wherein a spacing between the ultrasound transducer elements of the array of ultrasound elements is between about 0.01 mm and 2 mm;
control circuitry configured to operate the array of ultrasound transducer elements between 100 kHz and 1 MHz as a phased array to cause a beamforming of emitted ultrasound signals; and
a printed circuit board (PCB) substrate configured as a matching layer, onto which a front side of each ultrasound transducer element of the array of ultrasound transducer elements is mounted to transmit the emitted ultrasound signals through the PCB substrate, wherein the array of ultrasound transducers are air backed on a back side of each ultrasound transducer element;
wherein the control circuitry is on the PCB substrate.

46. The apparatus of claim 45, further comprising an outer surface of the apparatus configured to adhesively attach to a subject so that transmission of emitted ultrasound signals through the PCB is communicated to the subject's skin.

47. The apparatus of claim 45, further comprising an outer surface of the apparatus configured to be acoustically coupled, during the operation of apparatus to insonate a subject, with a subject's skin.

48. The apparatus of claim 45, wherein the width is between about 2 times and 10 times the thickness for each transducer element.

49. The apparatus of claim 45, wherein the width is approximately half of a wavelength of an ultrasound signal emitted by the apparatus.

50. The apparatus of claim 45, wherein the thickness is 25% or less of the wavelength of an ultrasound signal emitted by the device.
